(19) United States
(12) Patent Application Publication
(10) Pub. No.: US 2011/0136157 A1
(43) Pub. Date: Jun. 9, 2011

(54) Copper regulation evaluation and therapy

(76) Inventor: Garth James Smith Cooper, Auckland (NZ)

(21) Appl. No.: 11/919,051

(22) PCT Filed: Apr. 26, 2006
(86) PCT No.: PCT/NZ2006/000084

§ 371 Date: Apr. 12, 2010

Related U.S. Application Data
(60) Provisional application No. 60/674,734, filed on Apr. 25, 2005.
(60) Provisional application No. 60/675,310, filed on Apr. 26, 2005.

(51) Int. Cl. C12Q 1/60; C12Q 1/26; G01N 21/84

(52) U.S. Cl. 435/11; 435/25; 436/501

(57) ABSTRACT

Assays methods for clinical evaluation of conditions that might be treated with copper antagonists. These conditions include diabetes and other glucose metabolism disorders, lipid disorders, neurological disorders, and heart disease. The assays utilize a correlation between copper levels and one or more of the markers hemoglobin AIc and extracellular superoxide dismutase activity, in order to detect the condition, predict progression of the condition and assess a patient's response to copper antagonist therapy in these conditions by monitoring the level of these markers.

### FIELD OF THE INVENTION

The inventions relate generally to compositions containing a pharmaceutically acceptable copper antagonist compound, assays, assay methods and materials, and uses of the foregoing.

### BACKGROUND OF THE INVENTION

The following description includes information that may be useful in understanding the present invention. It is not an admission that any of the information provided herein is prior art, or relevant, to the presently described or claimed inventions, or that any publication or document that is specifically or implicitly referenced is prior art.

Diabetes mellitus is a glucose metabolism disorder and consists of a group of metabolic disorders associated with raised plasma glucose concentration and disturbance of glucose metabolism, which results in hyperglycemia. The World Health Organization (WHO) has set forth a classification scheme for diabetes mellitus that includes type 1 diabetes mellitus, type 2 diabetes mellitus, gestational diabetes, and other specific types of diabetes mellitus. Type 1 diabetes, also known as insulin-dependent diabetes mellitus, usually develops in children or young adults. Type 1 diabetes occurs when the pancreas produces too little insulin to regulate blood sugar levels appropriately. It is a chronic condition that ultimately requires daily insulin injections for survival. Although there is no set age, type 2 diabetes mellitus usually develops in people over 40 years old and is much more common that type 1 diabetes. Approximately 90% of all individuals with diabetes have type 2 diabetes. Type 2 diabetes mellitus is characterized by two different conditions: a decreased ability of insulin to act on peripheral tissues, usually referred to as “insulin resistance,” and dysfunction of pancreatic β-cells, represented by the inability to produce sufficient amounts of insulin to overcome insulin resistance in the peripheral tissues. Eventually, insulin production becomes insufficient to compensate for the insulin resistance due to β-cell dysfunction, which ultimately leads to β-cell failure. The result is a relative or absolute deficiency of insulin even though many people with type 2 diabetes for at least a period of time are hyperinsulinemic. Most patients with type 2 diabetes require pharmacotherapy, initially as monotherapy and subsequently in combination, as adjuncts to diet and exercise. Exogenous insulin is ultimately required in a substantial proportion, reflecting the progressive natural history of the disease. Sulphonylureas and biguanides have been employed for over four decades as oral antidiabetic agents, but they have a limited capacity to provide long term glycemic control and can cause serious adverse effects. Thus, more efficacious and tolerable antidiabetic agents are required.

In 2001, diabetes was the sixth leading cause of death in the United States. It is estimated that about 18 million people in the United States have diabetes, and over 5 million of these people are unaware that they have the disease. The Center for Disease Control (CDC) predicts that one in three Americans born in 2000 will develop diabetes during their lifetime. The total annual economic cost of diabetes in 2002 was estimated to be $132 billion, or one out of every 10 health care dollars spent in the United States. Center for Disease Control, The Burden of Chronic Diseases and Their Risk Factors (2004). The number of people with diabetes worldwide continues to increase at alarming rates. In 1985, it was estimated that 30 million people had diabetes. In 2000, the number was increased to 171 million. By 2030 the number of people suffering from diabetes worldwide is expected to reach 366 million. Wild et al., Diabetes Care 27(5):1047-1053 (2004).

Patients with diabetes have an increased incidence of long-term complications, which include atherosclerotic, cardiovascular, peripheral vascular, and cerebrovascular diseases. See American Diabetes Association, Diabetes Care 16:72-78 (1993). Principal risk factors for vascular complications have been discussed in relation to the degree and duration of hyperglycemia. The Diabetes Control and Complications Trial Research Group, N Engl J Med 329:977-986 (1993). Vascular complications can be divided into two groups, microvascular and macrovascular. In general, microvascular complications are said to affect the retina, kidney and nerves, while macrovascular complications are said to include diseases of the large vessels supplying the legs (lower extremity arterial disease), and predominantly coronary, cerebrovascular and peripheral arterial circulation. Chronic hyperglycemia of diabetes is associated with long-term damage, dysfunction, and failure of various organs, especially the eyes, kidneys, nerves, heart, and blood vessels. Long-term complications of diabetes include retinopathy with potential loss of vision; nephropathy leading to renal failure; peripheral neuropathy with risk of foot ulcers, amputation, and Charcot joints; and autonomic neuropathy causing gastrointestinal, genitourinary, and cardiovascular symptoms and sexual dysfunction.

While insulin resistance is a common factor leading to hyperglycemia in type 2 diabetes, it has also been reported that impaired glucose tolerance increases cardiovascular risk despite minimal hyperglycemia. Fuller J H, et al., Lancet 1:1373-1376 (1980). In the absence of diabetes, insulin resistance is reportedly a major risk factor for coronary artery disease (CAD). Lempiainen P, et al., Circulation 100:123-128 (1999). Insulin resistance coupled with compensatory hyperinsulinemia leads to a number of proatherogenic abnormalities referred to as Insulin Resistance Syndrome. Insulin Resistance Syndrome (also known as Metabolic Syndrome or Syndrome X) is a constellation of metabolic disturbances that enhance cardiovascular risk. Syndrome characteristics include deposition of fat around the abdominal organs, called visceral or central adiposity, changes in the lipoprotein profile, such as a decrease in HDL, a rise in triglycerides and an increase in low density lipoprotein (LDL). An increase in blood pressure is seen in many, but not all, insulin resistant populations. Increased fibrinogen, a clotting and inflammatory marker, and PAI-1, are also reported.

Heart disease is the leading cause of death for both women and men in the United States. In 2001, 700,142 people died of heart disease (52% of them women), accounting for 29% of all U.S. deaths. The age-adjusted death rate was 246 per 100,000 population. In 2001, heart disease cost the United States $193.8 billion in total health care costs. Heart disease is the major complication that leads to death in diabetes mellitus (Gu K, et al., Diabetes Care 21:1138-1145 (1998)) which, as noted above, is characterized both by chronic hyperglycemia and diffuse cardiovascular disease. Struthers A D, Morris A D, Lancet 359:1430-1432 (2002).

Obesity is one in a class of weight disorders. According to the WHO, obesity has reached epidemic proportions globally—with more than 1 billion adults overweight, at least 300 million of them clinically obese—and is a major contributor to the global burden of chronic disease and disability. Overweight conditions, including obesity, lead to adverse metabolic effects on body fat, cholesterol, triglycerides and insulin resistance and pose a major risk for chronic diseases. The likelihood of developing type 2 diabetes and hypertension rises steeply with increasing body fat. Confined to older adults for most of the 20th century, this disease now affects obese children even before puberty. Approximately 90% of people with type 2 diabetes are obese or overweight.

Wilson's disease, also known as hepatolenticular degeneration, is due to a defect in copper excretion into the bile by the liver. Wilson's disease occurs in individuals who have inherited an autosomal recessive defect that leads to an accumulation of copper in excess of metabolic requirements. The excess copper is deposited in several organs and tissues, and eventually produces pathological effects primarily in the liver, where damage progresses to postnecrotic cirrhosis, and in the brain, where degeneration is widespread. Copper is also deposited as characteristic, asymptomatic, golden-brown Kayser-Fleisher rings in the corneas of all patients with cerebral symptomatology and some patients who are either asymptomatic or manifest only hepatic symptomatology. Wilson's disease generally affects patients between the ages of 10 and 40 years.

Wilson's disease is generally treated with an orally administered copper chelator. First line therapy for treatment of Wilson's disease is penicillamine, a chelating agent. Penicillamine, 3-mercapto-D-valine, is also used to reduce cysteine excretion in cysteinuria and to treat patients with severe, active rheumatoid arthritis unresponsive to conventional therapy. It is a white or practically white, crystalline powder, freely soluble in water, slightly soluble in alcohol, and the empirical formula is C₅H₁₁NO₂S, giving it a molecular weight of 149.21. Cuprimine® (Penicillamine) capsules for oral administration contain either 125 mg or 250 mg of penicillamine, as well as D & C Yellow 10, gelatin, lactose, magnesium stearate, and titanium dioxide as inactive ingredients. The 125 mg capsule also contains iron oxide for capsule color. Triethylenetetramine dihydrochloride, also referred to as N,N′-bis(2-aminoethyl)-1,2-ethanediamine dihydrochloride, a chelating compound for removal of excess copper from the body, is prescribed for Wilson's disease patients who cannot tolerate penicillamine. It is a white to pale yellow crystalline hygroscopic powder that is freely soluble in water, soluble in methanol, slightly soluble in ethanol, and insoluble in chloroform and ether. The empirical formula is C₆H₁₈N₄.2HCl and it has a molecular weight of 219.2. The structural formula is NH₂(CH₂)₂—NH(CH₂)₂—NH(CH₂)₂—NH₂.2HCl. Sold in the United States under the tradename Syprine® triethylenetetramine hydrochloride is available as 250 mg capsules for oral administration. Syprine® capsules reportedly contain gelatin, iron oxides (for capsule color), stearic acid, and titanium dioxide as inactive ingredients. It has been reported that chelated copper in patients with Wilson's disease is excreted primarily through the feces, either by the effective chelation of copper in the gut, or by partial restoration of mechanisms that allow for excretion of excess copper via urine or into the bile, or a combination of the two. See Siegemund R, et al., Acta Neurol Scand. 83:364-6 (1991).

Zinc acetate (Galzin™) blocks the absorption of copper in the intestinal tract and was recently approved by the FDA for treatment of Wilson's disease. By blocking copper absorption, newly ingested copper does not reach the circulation and is excreted mainly in the stool. Zinc acetate has not shown any long-term or major side effects in patients and can be used, long-term, in place of non-tolerable chelating agents, which is useful for patients who develop adverse reactions to chelating agents.

Metal ions are essential for cells, but can become toxic at higher concentrations, and free metal ions have been implicated in heart disease. Metal ions can replace other essential metals in enzymes or molecules and disrupt their function. Metal ions such as Hg⁺ and Cu⁺ are reactive to thiol groups and can interfere with protein structure and function. Redox active transition metals such as Fe^{2+/3+} and Cu^{1+/2+}, which can take up or give off an electron, give rise to free radicals which can cause oxidative stress. Jones, et al., Biochim. Biophys. Acta 286:652-655 (1991); Li and Trush, Carcinogenes 7:1303-1311 (1993). Oxidative stress has been implicated as a factor in age-related disorders including diabetes mellitus, hypertension, obesity and atherosclerosis. Halliwell B, Gutteridge J M, Free Radicals in Biology and Medicine (University Press, 3^{rd} ed. 1999). However, clinical trials with antioxidants (MRC authors, Lancet 360:23-33 (2002)) or carbonyl-trapping agents (Monnier V M, J Clin Invest 107:799-801 (2001)) in these disorders have had mixed success. Aspects of the biology of transition metals including Zn, Mn, Mo, Cr, V, Fe and Cu, have been studied in the context of diabetes. Id. It was reported that free Fe and Cu ions are highly redox-active in mammalian tissues (Fraústo da Silva J J, Williams R J: The Biological Chemistry of the Elements: The Inorganic Chemistry of Life. 2nd ed. Oxford, U.K., Clarendon Press, 2001), where they may contribute to tissue damage by generation of reactive oxygen species (ROS) such as hydroxyl and peroxynitrite radicals. Halliwell (2001) supra; Kadiiska, M. B., et al., Mol Pharmacol 42:723-729 (1992). Although Cu is an essential trace nutrient, it is also a potent cytotoxin when excess accumulates in tissues. Peña M M, et al., J Nutr 129:1251-1260 (1999). However, the in vivo availability of catalytic Fe and Cu is usually very restricted, which acts as an important antioxidant defense. Fraústo da Silva (2001) supra.

Links between altered regulation of Fe metabolism and diabetes and heart disease have been reported in certain Fe regulation abnormalities. For example, altered homeostasis leading to Fe accumulation in the heart (Buja L M, Roberts W C, Am J Med 51:209-221 (1971) and pancreas has been related to cardiac disease and diabetes mellitus in hemochromatosis (Feder J N, et al., Nat Genet. 13:399-408 (1996)) and hemosiderosis. Telfer P T, et al., Br J Haematol 110:971-977 (2002). Fe and Cu were previously discussed in the context of the pathogenesis of diabetic complications, oxidative stress, and transition metal availability. Wolff S P, et al., Free Radic Biol Med 10:339-352 (1991). However, abnormalities of Fe homeostasis have not been linked to the major classes of diabetes mellitus, type 1 and type 2 diabetes, and whether there may be a role for Cu or alterations of Cu-metabolism in relation to the origins and progression of the complications of diabetes has remained unknown and, for the most part, unexplored.

It was recently reported that administration of triethylenetetramine caused a Cu(II)-triethylenetetramine complex to appear in the urine of STZ-diabetic rats. Cooper, G. J., et al., Diabetes 53:2501-2508 (2004). In diabetic animals with established heart failure, oral triethylenetetramine dihydrochloride for seven weeks alleviated heart failure, substantially improved cardiomyocyte structure and reversed elevations in left ventricular collagen and β₁ integrin, all without lowering blood glucose. Id. Oral triethylenetetramine dihydrochloride was also demonstrated to cause elevated Cu excretion in humans with type 2 diabetes, in whom treatment for six months led to a reduction in elevated left ventricular mass, implicating increased systemic accumulation of loosely-bound (chelatable) Cu(II) in the mechanism by which diabetes damages the heart. Id. No comparable link with Fe metabolism was detected. Id. See U.S. Pat. Nos. 6,610,693, 6,348,465, and 6,897,243 which provide copper chelators and other agents (e.g., zinc which prevents copper absorption) to decrease copper values for the benefit of subjects suffering from diabetes and its complications. See also, Cooper, G. J., et al., U.S. Pat. No. 6,951,890.

Extracellular superoxide dismutase (EC-SOD), a secretory glycoprotein, is the major superoxide dismutase (SOD) isoenzyme in extracellular fluids (Adachi T, et al., Clin Chim Acta 229:123-131 (1994)) and blood vessel walls (Fukai T, et al., Cardiovasc Res 55:239-249 (2002)). Activity and concentrations of serum EC-SOD have been reported to be elevated in subjects with diabetes (Adachi, T., et al., 1994, supra; Adachi, T., et al., J Endocrinol 181:413-417 (2004)), and the serum concentration of EC-SOD in relation to the severity of micro- and macrovascular diabetic complications have been discussed. Kimura F, et al., Diabetes Care 26:1246-1250 (2003). See Vivoli, G., et al., Biol Trace Elem Res 49:97-106 (1995) regarding [Cu]ₛₑᵣᵤₘ and EC-SOD activity in relation to humans with essential hypertension. While association of serum EC-SOD activity with the duration of diabetes, carotid artery intimal-media thickness, and severity of nephropathy and retinopathy has been discussed, and serum EC-SOD activity has been proposed as a marker of vascular injury (Kimura (2003) supra), erythrocyte SOD concentrations were reported to be essentially identical in normal subjects and patients with Wilson's disease, which is characterized by excess copper. Alexander, N. M., and Benson, G. D., Life Sciences 16:1025-1032 (1975).

A report on the effects of copper on in vitro gene expression concluded that copper can activate cholesterogenic genes in macrophages, where it was reported to increase expression of seven cholesterogenic genes, low-density lipoprotein receptor and HMG CoA reductase, and decrease the expression of CD36 and lipid binding proteins. Svensson, P. A., et al., Atherosclerosis 169:71-6 (2003). In patients with Wilson's disease, however, total cholesterol and LDL cholesterol were reported to be significantly lower when compared with control subjects. Rodo, M. et al., Eur J Neurol. 7:491-4 (2000).

Among other things, the inventions described and claimed herein include novel methods for the evaluation of subjects suffering from, or at risk for, one or more serious diseases, disorders or conditions, including heart disease, glucose metabolism disorders, weight disorders, lipid disorders, and neurological disorders, their prognosis, and their treatment with copper antagonist compounds. Also provided are novel methods for the reduction of superoxide in subjects in need thereof, as well as novel methods for the reduction of superoxide and EC-SOD in subjects in need thereof. These methods include, for example, methods for the reduction of superoxide and EC-SOD in people with diabetes and other glucose-metabolism diseases, disorders and conditions, as well as in subjects with other diseases, disorders and conditions characterized in whole or in part by increased levels of superoxide, EC-SOD, and/or EC-SOD activity. Additional methods include, for example, methods of increasing non-circulating EC-SOD, and methods of increasing arterial and cardiovascular EC-SOD.

### BRIEF DESCRIPTION OF THE INVENTION

The inventions described and claimed herein have many attributes and embodiments including, but not limited to, those set forth or described or referenced in this Summary. The inventions described and claimed herein are not limited to or by the features or embodiments identified in this Summary, which is included for purposes of illustration only and not restriction.

Methods for assessing subjects for copper regulation therapy are provided.

Methods for assessing candidate copper antagonist compounds for use in copper regulation therapy are also provided.

The inventions include methods of determining the probably response, or probability of response of a subject to a copper antagonist for treatment of a disease, disorder or condition, or evaluating the desirability of initiating, continuing, adjusting, or terminating copper regulation therapy in a subject, comprising making and/or correlating (i) a copper measurement or a measurement of copper in a sample from the subject and (ii) a hemoglobin A_{1c} measurement and/or a measurement of superoxide, or serum or plasma extracellular superoxide dismutase or extracellular superoxide dismutase activity, and identifying therefrom a response or a probability of response to copper antagonist treatment. Measurements may be actual or historical, and may be evaluated, for example, by reference to a table of variables or a figure such as that shown in FIG. 1. In one embodiment, for example, both hemoglobin A_{1c} and a measurement of superoxide, or serum or plasma extracellular superoxide dismutase are correlated. In another embodiment, a positive response probability is identified if (i) serum or urine copper is above normal levels and (ii) hemoglobin A_{1c} and/or superoxide or serum or plasma extracellular superoxide dismutase (measured by amount or activity) is/are above normal levels. In another embodiment, a positive response probability is identified if (i) serum copper is at least about 14 μM and (ii) hemoglobin A_{1c} is at least about 8% and/or (iii) superoxide is elevated or serum or plasma extracellular superoxide dismutase activity is at least about 1.5 times the upper limit of normal extracellular superoxide dismutase activity. In still another embodiment, a positive response probability is identified if (i) serum copper is at least about 20 μM and (ii) hemoglobin A_{1c} is at least about 6 to about 8% and/or (iii) superoxide is elevated or serum or plasma extracellular superoxide dismutase activity is at least about 1.5 times the upper limit of normal extracellular superoxide dismutase activity. In other embodiments, measures of urine copper are utilized. Measures of total copper, or copper balance, may also be used.

Also provided are methods of determining response of a subject to a copper antagonist for treatment of, for example, a disease, disorder or condition which is characterized in whole or in part by (a) hypercupremia and/or copper-related tissue damage and (b) one or more of hypertension, hyperlipidemia, impaired glucose tolerance, impaired fasting glucose, hyperglycemia, and insulin resistance, or predisposition to, or risk for, (a) and (b). Such diseases, disorders and/or conditions include but are not limited to those described or referenced herein.

In other embodiments, a measurement(s) of homocysteine is used in place of or in addition to a copper measurement(s). For example, the invention may include or further comprise identifying said subject as suitable for copper regulation therapy if said homocysteine is at least about 11.4 μM/L.

In other embodiments, other measures of glycemia is/are used in place of or in addition to an hemoglobin A_{1c} measurement(s), whether actual or historical. For example, the invention may include or further comprise identifying said subject as suitable for copper regulation therapy, or evaluating copper regulation therapy, by assessing glycemia using one or more fructoseamine measurement(s).

Also provided is a method of treating a subject having (a) a serum copper level of at least about 14 μM and/or a urine copper level of at least about 100 nM, (b) one or more of a hemoglobin A_{1c} of at least about 8% and a serum extracellular superoxide dismutase activity of at least about 40 Units per liter, comprising administering a therapeutically effective amount of a copper (II) antagonist to said subject.

Also provided is a method of treating a subject for heart disease, said subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper (II) antagonist to said subject.

Also provided is a method of treating a subject for a glucose metabolism disorder, said subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper (II) antagonist to said subject.

Also provided is a method of treating a subject for a weight disorder, said subject having (a) a serum copper level of at least about 14 μm and/or a urine copper level of at least about 100 nM and (b) a serum extracellular superoxide dismutase activity of at least about 40 Units per liter, comprising administering a therapeutically effective amount of a copper (II) antagonist to said subject.

Also provided is a method of treating a subject for a lipid disorder, said subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper (II) antagonist to said subject.

Also provided is a method of treating a subject for a neurological disorder, said subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper (II) antagonist to said subject.

Also provided is a method of treatment, comprising administering a therapeutically effective amount of a copper (II) antagonist to a subject with elevated oxidized LDL cholesterol. Also provided is a method of treating a subject with elevated oxidized LDL cholesterol, comprising administering a therapeutically effective amount of a copper (II) antagonist to said subject. Also provided is a method of treating a subject with elevated oxidized LDL cholesterol, comprising administering a therapeutically effective amount of a copper (H) antagonist to said subject. In some embodiments, the heart disease is selected from the group consisting of hypertension, atherosclerosis, heart failure, and cardiomyopathy. In some embodiments, said atherosclerosis is cerebrovascular atherosclerosis. In some embodiments, the glucose metabolism disorder is selected from the group consisting of impaired glucose tolerance, impaired fasting glucose, prediabetes, type 1 diabetes, type 2 diabetes, insulin resistance, hyperglycemia, hyperinsulinemia, hyperamylinemia, and metabolic syndrome. In some embodiments, the weight disorder is obesity. In some embodiments, the lipid disorder is selected from the group consisting of hyperlipidemia, hypertriglyceridemia, and hypercholesterolemia. In some embodiments, the neurological disorder is selected from the group consisting of Alzheimer's disease, Huntington's Disease and Parkinson's disease. In some embodiments, the copper antagonist is a linear or branched tetramine capable of binding copper. In other embodiments, the copper antagonist is selected from the group consisting of 2,3,2 tetramine, 2,2,2 tetramine, and 3,3,3 tetramine. In still other embodiments, the copper antagonist is a triethylenetetramine. In some embodiments, the copper antagonist is a triethylenetetramine salt. In some embodiments, the antagonist is a triethylenetetramine hydrochloride salt. In some embodiments, the triethylenetetramine hydrochloride salt is triethylenetetramine dihydrochloride. In other embodiments, the copper antagonist is a triethylenetetramine succinate salt. In some embodiments, the triethylenetetramine succinate salt is triethylenetetramine disuccinate. In other embodiments, the copper antagonist is pre-complexed with a non-copper metal ion.

Also provided are methods for the reduction of circulating superoxide or EC-SOD (e.g. EC-SOD as measured in plasma or serum) in mammalian subjects, including humans. These methods include methods for the reduction of superoxide or plasma EC-SOD in mammals, including humans, having diabetes or other glucose-metabolism diseases, disorders and conditions.

Also provided are methods for the reduction of superoxide or circulating EC-SOD in mammals, including humans, with diseases, disorders and conditions characterized in whole or in part by increased levels of superoxide or plasma or serum EC-SOD or EC-SOD activity, or in which modulation of superoxide and/or plasma EC-SOD would be beneficial.

Also provided are methods for increasing EC-SOD expression and methods for production of non-circulating EC-SOD (e.g. production of heparan sulfate bound EC-SOD). These methods include methods for the increase of non-circulating EC-SOD in mammals, including humans, with diseases, disorders and conditions characterized in whole or in part by decreased levels of arterial or cardiovascular EC-SOD concentration or activity, or in which modulation of arterial or cardiovascular EC-SOD would be beneficial.

Also provided are methods for increasing levels of heparan sulfate.

Assays capable of measuring chelatable copper are also provided.

Diseases, disorders and conditions that may be treated by the methods herein include, for example, heart diseases, glucose metabolism disorders, weight disorders, lipid disorders, and neurological disorders. Glucose metabolism disorders include, for example, impaired glucose tolerance, impaired fasting glucose, prediabetes, type 1 diabetes, type 2 diabetes, insulin resistance, hyperglycemia, hyperinsulinemia, hyperamylinemia, and metabolic syndrome. Heart diseases include, for example, hypertension, atherosclerosis, heart failure, and cardiomyopathy. Weight disorders include, for example, obesity. Lipid disorders include, for example, hyperlipidemia, hypertriglyceridemia, and hypercholesterolemia. Neurological disorders include, for example, Alzheimer's disease, Huntington's Disease and Parkinson's disease.

Subjects include humans and other mammals.

Other methods, including evaluation and therapeutic methods, are also provided and described and claimed herein.

### BRIEF DESCRIPTION OF THE DRAWINGS

FIG. 1 shows a Spline fine-grid response surface fitted to a 3-dimensional plot of the relationship between copper, as measured here by [Cu]ₛₑᵣᵤₘ (Oₓ), glycemia, as measured here by HbA_{1c} (O_{y}), and EC-SOD, as measured here by serum EC-SOD activity (O_{z}) in type-2 diabetic subjects (n=20) at baseline.

FIG. 2 shows restoration of EC-SOD mRNA levels in LV and aortic tissues from diabetic rats by 2.8- and 1.8-fold, respectively, following copper antagonist treatment (FIG. 2A-B).

FIG. 3A shows the heparan sulfate concentration in the left ventricle (LV) of untreated non-diabetic rats (N.D.); triethylenetetramine-treated non-diabetic rats (N.D.+T.); diabetic rats (Dia.); and triethylenetetramine-treated diabetic rats (Dia.+T.)(*P<0.05, **P<0.01, ***P<0.001).

FIG. 3B shows the heparan sulfate concentration in the aorta of untreated non-diabetic rats (N.D.); triethylenetetramine-treated non-diabetic rats (N.D.+T.); diabetic rats (Dia.); and triethylenetetramine-treated diabetic rats (Dia.+T.)(*P<0.05, **P<0.01, ***P<0.001).

### DETAILED DESCRIPTION OF THE INVENTION

A description of full copper balance in diabetes is provided, together with studies in which full 6-day balance of nine elements was measured in human subjects with type 2 diabetes and in age-matched non-diabetic controls. In a subsequent 6-day, 2×2 parallel group, placebo controlled study in the same subjects, systemic metal balance was then probed with a copper antagonist, triethylenetetramine.

Baseline urinary excretion of Cu and Fe was significantly increased in the diabetes group, and their δ values strongly correlated. Copper antagonism increased dose-dependent urinary excretion of Cu in a manner discovered to be predicted by baseline urinary Cu, thereby causing positive Cu balance to become negative in the diabetes group, whereas by contrast it modified neither Fe balance nor rates of urinary or fecal Fe excretion. Regulation of Cu metabolism was shown to be abnormal in the diabetes group and selectively modulated by copper antagonism, which acted without concomitant alteration of Fe metabolism. Copper antagonism did not alter the balance of any other element in either diabetic or control subjects.

Methods for assessing subjects for and during copper regulation therapy are provided.

Also provided are methods, for example, to assess the desirability or appropriateness of initiating, continuing, altering, or terminating copper regulation therapy.

Also provided are methods for lowering copper, particularly copper (II), in subjects described herein, comprising administering to said subjects a therapeutically effective amount of a copper antagonist, for example, a copper (II) antagonist. Methods for lowering superoxide are also provided.

Also provided are methods for elevating EC-SOD in tissues and/or lowering circulating serum or plasma EC-SOD in a subject, comprising administering to said subject a therapeutically effective amount of a copper antagonist, for example, a copper (II) antagonist. Also provided are methods for increasing arterial and/or cardiovascular EC-SOD in a subject, comprising comprising administering to said subject a therapeutically effective amount of a copper antagonist, for example, a copper (II) antagonist.

Also provided are methods for increasing EC-SOD expression, comprising administering to said subject a therapeutically effective amount of a copper antagonist, for example, a copper (II) antagonist. In some embodiments, said methods increase arterial and/or cardiovascular EC-SOD expression.

Also provided are methods for increasing heparan sulfate levels in a subject, comprising administering to said subject a therapeutically effective amount of a copper antagonist, for example, a copper (II) antagonist, whereby heparan sulfate levels are increased.

Also provided are methods for lowering superoxide in a subject, comprising administering to said subject a therapeutically effective amount of a copper antagonist, for example, a copper (II) antagonist.

As used herein, a “copper antagonist” is a pharmaceutically acceptable compound that binds or chelates copper, preferably copper (II), in vivo. Copper chelators are presently preferred copper antagonists. Copper (II) chelators, and copper (ID-specific chelators (i.e., those that preferentially bind copper (II) over other forms of copper such as copper (I)), are especially preferred. Copper antagonism may be evaluated by assessing urinary copper, as disclosed herein, for example. Copper antagonism may also be evaluated, by way of further example, by assessing serum copper, total copper, or copper balance. Both historical and actual measures of copper antagonism may be used in the methods of the invention.

“Copper regulation therapy” refers to the use of copper antagonists, preferably copper (II) antagonists, for example, copper (II) chelators, for the treatment of a subject in need thereof.

The term “copper” as used herein in reference to the use of diagnostic and prognostic indicators, for example, is not intended to be limited to a particular form of copper. Rather it refers to all forms of copper in a patient, which may include Cu(I), Cu(II), and total copper. “Copper (II)” refers to the oxidized (or +2) form of copper, also sometimes referred to as Cu⁺².

As used herein, a “pre-complexed copper antagonist” is a pharmaceutically acceptable compound wherein a copper antagonist, for example a copper chelator, has been pre-complexed with a non-copper metal ion prior to administration for therapy. Metal ions used for pre-complexing have a lower association constant for the copper antagonist than that of copper. Pre-complexed copper antagonists may be prepared for administration via oral delivery.

The term “correlating” as used herein in reference to the use of diagnostic and prognostic indicators, refers to comparing the presence or amount of the indicator in a patient to its presence or amount in persons known to respond to a certain treatment, suffer from, or known to be at risk of, a given condition, or in persons known to be free of a given condition, e.g., “normal individuals.” Measurements from patient samples may be used, as may historical or actual measurements.

The term “diagnosis” as used herein refers to methods by which the skilled artisan can estimate and or determine whether or not a patient is suffering from a given disease or condition. The skilled artisan often makes a diagnosis on the basis of one or more diagnostic indicators, the presence, absence, or amount of which is/are indicative of the presence, severity, or absence of the condition.

Similarly, a prognosis is often determined by examining one or more “prognostic indicators.” These include biomarkers, for example, the presence or amount of which in a patient (or a sample obtained from the patient) signal a probability that a given course or outcome, including treatment outcome, will occur. For example, when one or more prognostic indicators exhibit a certain pattern or level in samples obtained from such patients, the pattern or level may signal that the patient is at an increased probability for experiencing a future event in comparison to a similar patient exhibiting a different pattern or lower marker level. A certain pattern, level or a change in level of a prognostic indicator, which in turn is associated with an increased probability of disease recurrence or side effect, such as obesity, is referred to as being “associated with an increased predisposition to an adverse outcome” in a patient. Preferred prognostic markers can predict the onset of delayed adverse events in a patient, or the chance of a person responding or not responding to a certain drug. The inventions include the use of copper (e.g., copper (II)), glycemia (e.g., hemoglobin A_{1c}), superoxide and/or serum or plasma EC-SOD (e.g., serum or plasma EC-SOD activity) as prognostic indicators.

The phrase “determining the prognosis” as used herein refers to methods by which the skilled artisan seeks to predict the course or outcome of a condition in a patient. The term “prognosis” does not refer to the ability to predict the course or outcome of a condition with 100% accuracy, or even that a given course or outcome is predictably more or less likely to occur based on the presence, absence or levels of test markers. Instead, the skilled artisan will understand that the term “prognosis” refers to an increased probability that a certain course or outcome will occur; that is, that a course or outcome is more likely than not to occur in a patient exhibiting a given condition, such as diabetes or heart disease, when compared to those individuals not exhibiting the condition. The inventions include the use of copper (e.g., copper (II)), glycemia (e.g., hemoglobin A_{1c}), superoxide and/or serum or plasma EC-SOD (e.g., serum or plasma EC-SOD activity) in determining the prognosis of a patient or therapy.

As used herein, a “disorder” is any disorder, disease, or condition that would benefit from (1) an agent that reduces superoxide, (2) an agent that increases arterial or cardiovascular EC-SOD and/or EC-SOD activity, (3) an agent that reduces serum or plasma EC-SOD and/or EC-SOD activity, (4) an agent that reduces local or systemic copper, extracellular copper, bound copper, copper concentrations, total copper, or copper balance, and/or (5) an agent that reduces glycemia, for example. Particularly preferred are agents that reduce serum or plasma EC-SOD and/or EC-SOD activity. Also particularly preferred are agents that reduce extracellular copper or extracellular copper concentrations (local or systemic) and, more particularly, agents that reduce extracellular copper (II) or extracellular copper (II) concentrations (local or systemic), including agents that reduce total copper (sometimes referred to as copper values) or lower copper balance. Disorders include, but are not limited to, those described and/or referenced herein, and include diseases, disorders and conditions include that would benefit from (1) a decrease in body and/or tissue copper levels, including serum copper levels, (2) an increase copper output in the urine, (3) a decrease in copper uptake, for example, in the gastrointestinal tract, (4) a decrease in copper balance, (5) a decrease in SOD, for example, serum or plasma EC-SOD, as measured by mass or activity, (6) decreased glycemia (e.g., a decrease in serum glucose, (7) a decrease in blood glucose, (8) a decrease in urine glucose, (9) a decrease in fructosamine, (10) a decrease in glycosylated hemoglobin (HbA_{1c}) levels, (11) a decrease in postprandial glycemia, (12) an improvement in impaired glucose tolerance, (13) an improvement in impaired fasting glucose, (14) a in decrease weight, (15) a decrease in the rate and/or severity of hypoglycemic events, including severe hypoglycemic events, (16) a decrease in hyperlipidemia (including, for example, hypercholesterolemia and hypertriglyceridemia), (17) a decrease in blood pressure, (18) improved cardiovascular function, (19) increased arterial or cardiovascular EC-SOD or EC-SOD activity, and/or (20) increased heparan sulfate.

Such disorders include, for example, but are not limited to, glucose metabolism disorders; cardiovascular disorders; neurodegenerative disorders; insulin disorders; liver disorders; lipid/cholesterol disorders; diseases, disorders, and conditions treated or treatable with insulin; diseases, disorders, and conditions treated or treatable with hypoglycemic agents; diseases, disorders, and conditions treated or treatable with statins and the like; diseases, disorders, and conditions treated or treatable with antihypertensive agents; diseases, disorders, and conditions treated or treatable with anti-obesity agents; diseases, disorders or conditions treated or treatable with biologically active protein C or a protein C derivative; and diseases, disorders, and conditions treated or treatable with copper antagonists including, for example, copper (II) chelators.

Diseases, disorders and conditions that may be evaluated, prevented, treated or ameliorated include, for example, atherosclerosis; peripheral vascular disease; cardiovascular disease; heart disease; coronary heart disease; restenosis; angina; ischemia; heart failure; stroke; impaired glucose tolerance; impaired fasting glucose; prediabetes; diabetes and/or its complications, including type 1 and type 2 diabetes and their complications; insulin resistance; glucose metabolism diseases and disorders; chronic hepatitis; fatty liver disease, including non-alcoholic and alcoholic fatty liver disease; steatohepatitis, including non-alcoholic and alcoholic steatohepatitis, and other conditions involving inflammation of the liver; Syndrome X; obesity and other weight related disorders; cardiomyopathy, including diabetic cardiomyopathy; hyperglycemia; hypercholesterolemia (e.g., elevated cholesterol in low-density lipoprotein (LDL-C)); pre-hypertension, hypertension, secondary hypertension, malignant hypertension, isolated systolic hypertension, and portal hypertension; hyperinsulinemia; hyperlipidemia; Alzheimer's disease and Parkinson's disease; degenerative diseases; lupus and arthritis; nerve disease, including diabetic neuropathy; kidney disease, including diabetic nephropathy; eye disease, including diabetic retinopathy and cataracts; acute coronary syndromes, including myocardial infarction; vascular occlusive disorders; diseases, disorders associated with a hypercoagulable state or protein C deficiency, including but not limited to arterial thrombosis, arterial embolism, pulmonary embolism, deep venous thrombosis, venous thrombosis, renal vein thrombosis, mesenteric vein thrombosis, atheroembolic renal disease, thrombophlebitis, stroke, heart attack or angina, viral hemorrhagic fever, disseminated intravascular coagulation, purpura fulminans, bone marrow and other transplantations, severe burns, major surgery, severe trauma, adult respiratory distress syndrome, postphlebic syndrome, coumarin-induced skin necrosis; thrombotic diseases, disorders or conditions; sepsis and related diseases, disorders or conditions; diseases, disorders or conditions relating to undesired inflammation; thrombotic or embolic complications related to diseases, disorders or conditions including, but not limited to, diabetes, hypertension, pre-hypertension, portal hypertension, hyperlipidemia, hypercholesteremia, and/or atherosclerosis.

The invention also includes methods for evaluating and/or treating or preventing or ameliorating, in whole or in part, various diseases, disorders and conditions, including, for example, disorders of the heart muscle, including heart failure; myocardial infarction; cardiomyopathy, including idiopathic cardiomyopathy, metabolic cardiomyopathy, alcoholic cardiomyopathy, drug-induced cardiomyopathy, ischemic cardiomyopathy, and hypertensive cardiomyopathy. Other disorders that may be evaluated, treated or prevented, in whole or in part, according to the methods of the invention include diabetic acute coronary syndrome (e.g., myocardial infarction, diabetic hypertensive cardiomyopathy, acute coronary syndrome associated with impaired glucose tolerance (IGT), acute coronary syndrome associated with impaired fasting glucose (IFG), hypertensive cardiomyopathy associated with IGT, hypertensive cardiomyopathy associated with TEG, ischemic cardiomyopathy associated with IGT, ischemic cardiomyopathy associated with IFG, ischemic cardiomyopathy associated with coronary heart disease (CHD), acute coronary syndrome not associated with any abnormality of the glucose metabolism, hypertensive cardiomyopathy not associated with any apparent abnormality of glucose metabolism, ischemic cardiomyopathy not associated with any apparent abnormality of glucose metabolism (irrespective of whether or not such ischemic cardiomyopathy is associated with coronary heart disease or not), and any disease of the vascular tree including disease states of the aorta, carotid, cerebrovascular, coronary, renal, retinal, vasa nervorum, iliac, femoral, popliteal, arteriolar tree and capillary bed). Additionally, atheromatous disorders of the major blood vessels (including the aorta, the coronary arteries, the carotid arteries, the cerebrovascular arteries, the renal arteries, the iliac arteries, the femoral arteries, and the popliteal arteries), toxic, drug-induced, and metabolic disorders of small blood vessels, and, non-fatal plaque rupture of atheromatous lesions of major blood vessels, all may be evaluated, treated or prevented, in whole or in part, according to methods of the invention.

Diseases, disorders and conditions that may be that may be evaluated, prevented, treated or ameliorated also include, for example, (1) diseases, disorders and conditions characterized in part by any one or more of hyperlipidemia, hypercholesterolemia, hyperglycemia, hypertension, and/or hyperinsulinemia; (2) diseases, disorders or conditions characterized in whole or in part by (a) hypercupremia and/or copper-related tissue damage and (b) hyperglycemia, insulin resistance, impaired glucose tolerance, and/or impaired fasting glucose, and/or elevated or undesired levels of LDL-C, or predisposition to, or risk for, (a) and (b); (3) diseases, disorders and conditions characterized in whole or in part by (a) excess copper and/or copper-related tissue damage and (b) a BMI from about 25 to about 29.9 or a BMI greater than about 30 (including subjects having a BMI from about 30 to about 34.9 (obesity class I), from about 35 to 39.9 (obesity class II), and greater than about 40 (obesity class III)); (4) diseases, disorders or conditions characterized in whole or in part by (a) excess copper and/or copper-related tissue damage, and (b) protein C deficiency and/or undesired coagulation activity, or predisposition to, or risk for, (a) and (b); (5) diseases, disorders or conditions characterized in whole or in part by (a) excess copper and/or copper-related tissue damage, and (b) excess body fat; and subjects within the World Health Organization (WHO) classification for overweight and obesity (including subclassifications based on race and waist circumference), who are at risk for comorbid conditions, including hypertension, type 2 diabetes mellitus, and cardiovascular disease.

The invention includes methods for evaluating and/or treating a subject having or suspected of having or predisposed to, or at risk for, for example, any diseases, disorders and/or conditions described or referenced herein. A pharmaceutically acceptable copper antagonist and/or a pre-complexed copper antagonist may be administered in amounts, for example, that are effective to (1) decrease body and/or tissue copper levels, (2) increase copper output in the urine of said subject, (3) decrease copper uptake, for example, in the gastrointestinal tract, (4) lower serum or plasma EC-SOD, and/or (5) increase arterial and/or cardiovascular EC-SOD. Such compositions include, for example, tablets, capsules, solutions and suspensions for parenteral and oral delivery forms and formulations.

The invention includes methods for administering a therapeutically effective amount of a pharmaceutically acceptable copper antagonist and/or a pre-complexed copper antagonist in a delayed release preparation, a slow release preparation, an extended release preparation, a controlled release preparation, and/or in a repeat action preparation. Such preparations may be administered to a subject having or suspected of having or predisposed to diseases, disorders and/or conditions referenced herein. Such compounds may be administered in amounts, for example, that are effective to (1) decrease body and/or tissue copper levels, (2) increase copper output in the urine of said subject, (3) decrease copper uptake, for example, in the gastrointestinal tract, (4) lower serum or plasma EC-SOD, and/or (5) increase arterial cardiovascular EC-SOD, and/or (6) increase heparan sulfate. Such compositions include, for example, tablets, capsules, solutions and suspensions for parenteral and oral delivery forms and formulations.

As used herein, “mammal” refers to any animal classified as a mammal, including humans, domestic and farm animals, and zoo, sports, or pet animals, such as dogs, horses, cats, sheep, pigs, cows, etc. The preferred mammal herein is a human.

As used herein, “pharmaceutically acceptable salts” refers to salts prepared from pharmaceutically acceptable non-toxic bases or acids including inorganic or organic bases and inorganic or organic acids the like. When the copper antagonist compound is basic, salts may be prepared from pharmaceutically acceptable non-toxic acids, including inorganic and organic acids. Organic acids include both aliphatic and aromatic carboxylic acids and include, for example, aliphatic monocarboxylic acids, aliphatic dicarboxylic acids, aliphatic tricarboxylic acids, aromatic monocarboxylic acids, aromatic dicarboxylic acids, aromatic tricarboxylic acids and other organic acids known to those of skill in the art. Aliphatic carboxylic acids may be saturated or unsaturated. Suitable aliphatic carboxylic acids include those having from 2 to about 10 carbon atoms. Aliphatic monocarboxylic acids include saturated aliphatic monocarboxylic acids and unsaturated aliphatic monocarboxylic acids. Examples of saturated monocarboxylic acids include acetic acid, propronic acid, butyric acid, valeric acid, caproic acid, enanthic acid, caprylic acid, pelargonic acid, and caprynic acid. Examples of unsaturated aliphatic monocarboxylic acids include acrylic acid, propiolic acid, methacrylic acid, crotonic acid and isocrotonic acid. Aliphatic dicarboxylic acids include saturated aliphatic dicarboxylic acids and unsaturated aliphatic dicarboxylic acids. Examples of saturated aliphatic dicarboxylic acids include oxalic acid, malonic acid, succinic acid, glutaric acid, adipic acid, pimelic acid, suberic acid, azelaic acid, and sebacic acid. Examples of unsaturated aliphatic dicarboxylic acids include maleic acid, fumaric acid, citraconic acid, mesaconic acid, itaconic acid and the like. Aliphatic tricarboxylic acids includes saturated aliphatic tricarboxylic acids and unsaturated tricarboxylic acids. Examples of saturated tricarboxylic acids include tricarballylic acid, 1,2,3-butanetricarboxylic acid and the like. Suitable aliphatic dicarboxylic acids include those of the formula: HOOC-Q₁-COOH, wherein Q₁ is alkylene of 1 to about 8 carbon atoms or alkenylene of 2 to about 8 atoms, and includes both straight chain and branched chain alkylene and alkenylene groups. Examples of aromatic dicarboxylic acids include phthalic acid, isophthalic acid, terephthalic acid and the like. Examples of aromatic tricarboxylic acids include trimesic acid, hemimellitic acid and trimellitic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are hydrochloric, maleic, fumaric, and succinic acids. Succinic acid is most preferred.

As used herein, “preventing” means preventing in whole or in part, or ameliorating or controlling.

The term “test sample” as used herein refers to a biological sample obtained for the purpose of diagnosis, prognosis, or evaluation. In certain embodiments, such a sample may be obtained for the purpose of assessing or determining the outcome of an ongoing condition or assessing or determining the effect of a treatment regimen on a condition. Preferred test samples include blood, serum, plasma, cerebrospinal fluid, urine and saliva. In addition, one of skill in the art would realize that some test samples would be more readily analyzed following a fractionation or purification procedure, for example, separation of whole blood into serum or plasma components.

As used herein, a “therapeutically effective amount” in reference to compounds or compositions refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will generally involve the prevention, decrease, or reversal of effects relating to unwanted copper or copper levels, in whole or in part, undesired EC-SOD activity or levels, for example, increased plasma or serum EC-SOD, reduced EC-SOD, and/or reduced heparan sulfate as referenced herein. Therapeutic effects include those noted above and elsewhere herein, for example.

As used herein, the term “treating” refers to both therapeutic treatment and prophylactic or preventative measures. Those in need of treatment include those already with the disorder as well as those prone to having the disorder or diagnosed with the disorder or those in which the disorder is to be prevented.

Reductions in copper, particularly extracellular copper that is generally in the copper II form, serum or plasma EC-SOD (and/or an increase in arterial and/or cardiovascular EC-SOD), and/or an increase in heparan sulfate will be advantageous in the treatment of disorders, diseases, and/or conditions, caused or exacerbated by mechanisms that may be affected by, dependent on, or characterized by excess copper and/or circulating EC-SOD, and/or increased superoxide. For example, a reduction in copper, circulating EC-SOD (and/or an increase in arterial and/or cardiovascular EC-SOD) and/or an increase in heparan sulfate will be advantageous in providing a combined reduction in and/or reversal of copper-associated and/or superoxide-associated damage.

The discoveries described and claimed herein are directed, for example, to novel methods for the testing and evaluation of subjects suffering from, or at risk for, one or more serious diseases, disorders or conditions, including heart disease, cardiovascular disorders, vascular disorders, glucose metabolism disorders, weight disorders, lipid disorders, and neurological disorders, and their treatment with copper antagonist and/or a pre-complexed copper antagonist compounds.

Also disclosed and described herein are novel methods of treatment, including, for example, methods of reducing plasma or serum EC-SOD; methods of increasing tissue EC-SOD, (e.g. arterial and/or cardiovascular EC-SOD) and/or methods of increasing heparan sulfate, comprising administration of a copper antagonist to a subject.

As set forth in the Examples, homeostasis of copper (Cu) and eight other elements (iron, zinc, calcium, magnesium, manganese, molybdenum, selenium and chromium EC-SOD) in diabetes was characterized by measuring blood parameters and baseline 6-day elemental intakes, losses, and balances under fully residential conditions in male human subjects with type 2 diabetes (n=20) and age-matched controls (n=20). Elemental balance with an oral copper antagonist (triethylenetetramine dihydrochloride; 2.4 g/day) was probed in a parallel group, using a placebo-controlled factorial study in the same subjects over the following 6-days.

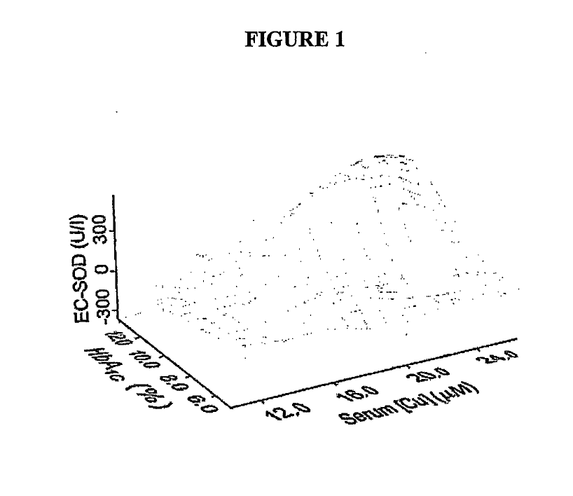

At baseline, there were no detectable between-group differences in the balance of any element, although urinary excretion of copper, iron, zinc, manganese, selenium and chromium was greater in diabetic subjects than controls. Mean circulating extracellular superoxide dismutase (EC-SOD) activity was elevated in diabetes and its activity correlated strongly with the interaction between [Cu]ₛₑᵣᵤₘ and hemoglobin A_{1c}. See FIG. 1.

In diabetic subjects, copper antagonist treatment with triethylenetetramine dihydrochloride caused copper balance to become negative through enhanced urinary copper excretion and suppressed elevated circulating EC-SOD, but did not modify balances of other elements in either control or diabetic subjects. Urinary copper losses during triethylenetetramine dihydrochloride treatment led to selective extraction of systemic Cu(II), thereby reversing the tendency for increased accumulation of loosely-bound Cu(II).

The Examples show regulation of copper homeostasis in diabetic human subjects before and after administration of a copper antagonist, here triethylenetetramine dihydrochloride, a Cu(II)-selective chelator. These studies show that several aspects of copper metabolism are altered in diabetic humans compared with matched control subjects. At baseline, urinary copper excretion was 1.4-fold higher in diabetic subjects, although [Cu]ₛₑᵣᵤₘ did not significantly differ from values in control subjects. See Smith R. G., et al., J Trace Elem Electrolytes Health Disease 2:239-243 (1988); Ito S, et al., Nephron 88:307-312 (2001). But see Walter R M Jr, et al., Diabetes Care 14:1050-1056 (1991).

There was a non-significant trend for copper balance to be elevated in type 2 diabetes compared with controls, as well as a significant interaction between copper regulation therapy and diabetes on copper balance. Copper antagonism via triethylenetetramine dihydrochloride treatment markedly stimulated urinary copper excretion compared with placebo in both diabetic and control subjects, but lowered copper balance only in diabetes.

Urinary copper excretion during copper antagonist administration was strongly and positively correlated with baseline (pretreatment) urinary copper excretion. Thus, elevated basal urinary copper predicted copper antagonist-induced cupruresis and individual responses may be determined by prior systemic Cu⁺² accumulation. By contrast, copper antagonism significantly decreased fecal copper excretion in control subjects only, possibly through increased uptake. Regulation of copper homeostasis differed significantly between diabetic and control subjects. Copper antagonism elicited specific effects to reverse elevated copper balance in diabetes, mainly through stimulation of urinary copper excretion.

The Examples herein indicate that measurements of normal [Cu]ₛₑᵣᵤₘ or [ceruloplasmin] are not likely to be informative concerning the likely presence of copper excess in diabetic subjects. Ceruloplasmin (ferro-O₂-oxidoreductase, EC 1.16.3.1) is a circulating copper protein found in vertebrate plasma that belongs to the family of multi-copper oxidases that also include ascorbate oxidase and laccases. Vachette, P., et al., J Biol Chem 277:40823-40831 (2002). Normally, more than 95% of plasma copper is bound to ceruloplasmin, and levels of circulating copper and ceruloplasmin are closely related. Hellman, N. E., Gitlin, J. D., Annu Rev Nutr 22:439-458 (2002). Plasma copper and ceruloplasmin levels are the frequently employed as measures of copper status, and are depressed in severe copper deficiency. Kehoe, C. A., et al., J Nutr 130:30-33 (2000). However, levels plateau when copper intake is adequate, and they do not reflect the magnitude of copper intake beyond this point, and thus unlikely to be useful for characterization of copper excess states. Hambidge M., J Nutr 133:948 S-955S (2003).

The Examples also show that serum EC-SOD activity was significantly elevated in diabetic subjects compared with controls. Serum EC-SOD activity was also strongly and positively correlated with an interaction between HbA_{1c} and [Cu]ₛₑᵣᵤₘ. This relationship is consistent with a mechanism whereby elevations in EC-SOD are caused by an interaction between [Cu]ₛₑᵣᵤₘ and chronic hyperglycemia. As the major SOD isoform present in vascular endothelium, where it acts to regulate superoxide levels, EC-SOD is a key regulator of endothelium-derived nitric oxide bioactivity in blood vessels. The elevation of serum EC-SOD reflects increased superoxide production in diabetes. Nishikawa, T., et al., Nature 404:787-791 (2000).

The decrease following copper antagonist treatment shown in the Examples is consistent with suppression of vascular superoxide production by copper antagonism, particularly Cu⁺² antagonism, and the results support an interaction between [Cu]ₛₑᵣᵤₘ and chronic hyperglycemia in the mechanism by which diabetes causes tissue damage. Six days of triethylenetetramine dihydrochloride treatment reversed elevated serum EC-SOD in the diabetic subjects to control values. The Examples show the utility of copper antagonism using, for example, a copper II antagonist, for lowering superoxide and lowering circulating EC-SOD.

Indices of copper balance were also compared with those of eight other elements in diabetic and control subjects, namely, calcium, magnesium, iron, manganese, molybdenum, selenium and chromium, all of which save Cr are essential nutrients. Subcommittee on the Tenth Edition of the RDAs Food and Nutrition Board Commission on Life Sciences, National Research Council: Recommended Dietary Allowances 10th ed. Washington D.C., U.S.A., National Academy Press, (1989). At baseline, elemental balance did not differ significantly between diabetic and control subjects.

Urinary excretion rates for iron, zinc, manganese, calcium, selenium and chromium were significantly elevated in diabetes, whereas fecal excretion rates were equivalent between the two groups. Triethylenetetramine dihydrochloride treatment significantly increased zinc balance in control but not diabetic subjects, mainly via suppression of fecal zinc excretion, whereas it stimulated urinary zinc in both groups. The effect on fecal zinc is consistent with drug-mediated increased uptake from the gut.

Previously, findings of hyperzincuria and low zinc absorption in diabetic animals and humans have prompted conjecture that diabetic subjects may be more susceptible to zinc deficiency. Escobar, O., et al., Pediatr Res 37:321-327 (1995). However, an earlier commentary reported increased tissue zinc values in animals following triethylenetetramine dihydrochloride treatment. Keen, C. L., et al., Proc Soc Exp Biol Med 173:598-605 (1983). Apparent absorption and retention of zinc and copper in rats fed a purified diet was measured in a balance study following induction of STZ-diabetes, in which food consumption was twice that of controls. Fractional absorption of zinc and copper was reportedly lower in the diabetic rats, but net absorption was higher, which was offset by higher urinary excretion, so that ultimate zinc and copper retention was similar in both groups. Id. Low fractional absorption has been attributed to lower rates of intestinal zinc transport, associated with increased concentrations of intestinal metallothionein, an inhibitor of zinc transport. Escobar (1995) supra.

Consistent with previous reports (Ford, E. S., Cogswell, M. E., Diabetes Care 22:1978-1983 (1999)), mean serum ferritin was increased in diabetic subjects, although other measures of iron homeostasis including serum iron and IBC (Cutler, P., Diabetes 38:1207-1210 (1989)) were not different from control values. Basal urinary iron excretion was also elevated in diabetic subjects, whereas by contrast, fecal iron output and iron balance were similar. Basal urinary iron excretion was closely correlated with increased basal urinary excretion of other divalent cations, including copper, zinc, manganese, selenium and chromium. Elevated urinary iron excretion in type 2 diabetes thus occurred in conjunction with elevated excretion of several other urinary elements. Triethylenetetramine dihydrochloride had no effect on indices of iron balance in diabetes, although it did increase iron balance in control subjects, mainly through suppression of fecal iron excretion.

The effects of copper antagonism on calcium, manganese and selenium were similar to those for iron. Others have reported that serum ferritin is elevated in a subset of subjects with poorly controlled type 2 diabetes with no known iron-storage disorders. Fernández-Real J M, et al. Diabetes Care 25:2249-2255 (2002). Treatment with the iron-selective chelator, deferoxamine, reportedly lowered increased ferritin levels and correlated with improved fasting glucose, triglyceride and HbA_{1c}, values (Escobar (1995)), but these findings were not replicated in subsequent studies (Kaye T B et al.,: J Diabetes Complications 7:246-249 (1993); Redmon et al., Diabetes 42:544-549, (1993). Regarding iron depletion by blood-letting in relation to HbA_{1c}, increased insulin sensitivity and islet β-cell function, and improved indices of vascular dysfunction in similar subjects, see Fernández-Real J M et al., Diabetes 51:1000-1004 (2002). At this time, however, the significance of high serum ferritin concentrations in a subset of patients with type 2 diabetes remains uncertain. Van Oost B A, et al., Clin Biochem 17:263-269 (1984). In the Third National Health and Nutrition Examination Survey (1988-1994), the finding that the increased risk of diabetes was concentrated among participants with the lowest transferrin saturation concentrations was interpreted to indicate that the iron overload hypothesis is unlikely to explain this association, and may instead implicate inflammation.

Thus, regulation of copper homeostasis is altered in diabetic subjects, who demonstrate elevated rates of urinary copper excretion and a tendency to increased copper balance compared with controls. Treatment with copper antagonists, for example a Cu(II)-selective chelator, such as triethylenetetramine dihydrochloride, can lower copper balance in diabetes by extraction of copper from the body.

Elevated serum EC-SOD was also strongly correlated with the interaction between [Cu]ₚₗₐₛₘₐ and chronic hyperglycemia. Treatment with copper antagonists, for example a Cu(II)-selective chelator, such as triethylenetetramine dihydrochloride, was also demonstrated to lower serum EC-SOD and to suppress elevations in serum EC-SOD.

Example 5 shows that EC-SOD mRNA expression was significantly decreased in the left ventricle and aorta of diabetic rats compared to non-diabetic rats. Treatment with a copper antagonist, for example a Cu(II)-selective chelator, such as triethylenetetramine dihydrochloride, was shown to normalize EC-SOD mRNA expression.

Example 6 shows that heparan sulfate levels are significantly decreased in the left ventricle and aorta of diabetic rats compared to levels in control rats. This example also demonstrates that treatment with a copper antagonist, for example a Cu(II)-selective chelator, such as triethylenetetramine dihydrochloride, significantly increased heparan sulfate levels in diabetic rats.

Provided are methods of determining response of a subject to a copper antagonist for treatment of a disease, disorder or condition, the method comprising: correlating (i) a measurement of copper in a sample from the subject with (ii) a hemoglobin A_{1c} measurement for the subject and/or a measurement of extracellular superoxide dismutase activity in a sample from the subject, and identifying therefrom the probability of response to the copper antagonist. In one embodiment, both hemoglobin A_{1c} and a measurement of extracellular superoxide dismutase (and/or superoxide) are correlated. In another embodiment, a positive response probability is identified if (i) serum copper is at least about 14 μM and (ii) hemoglobin A_{1c} is at least about 8% and/or plasma or serum extracellular superoxide dismutase activity is at least about 1.5 times the upper limit of normal extracellular superoxide dismutase activity. In another embodiment, a positive response probability is identified if (i) serum copper is at least about 14 μM, (ii) hemoglobin A_{1c} is at least about 8%, and (iii) plasma or serum extracellular superoxide dismutase activity is at least about 1.5 times the upper limit of normal extracellular superoxide dismutase activity. In another embodiment, a positive response probability is identified if (i) serum copper is at least about 20 μM and (ii) hemoglobin A_{1c} is at least about 6 to about 8% and/or (iii) plasma or serum extracellular superoxide dismutase activity is at least about 1.5 times the upper limit of normal extracellular superoxide dismutase activity. In yet another embodiment, extracellular superoxide dismutase activity is determined by measuring the amount of extracellular superoxide dismutase.

Copper, hemoglobin A_{1c}, extracellular superoxide dismutase activity, and superoxide levels can be measured by means known in the art or as described herein. For example, copper levels in a test sample may be measured using atomic adsorption spectroscopy (AAS), inductively coupled plasma-atomic emission spectroscopy (ICP-AES), differential-pulse anodic stripping voltammetry techniques, or by use of assays such at those described herein in Example 4. Extracellular superoxide dismutase activity may be measured, for example, be measuring protein mass, protein expression, or by using enzymatic assays such as measuring NAD(P)H oxidase activity. Superoxide is generated by molecular oxygen in the presence of EDTA, MnCl₂, and mercaptoethanol. Superoxide oxidizes NAD(P)H at a predictable rate and thereby lowers its absorbance at 340 nm. The decrease in absorbance is inhibited in the presence of SOD. Thus EC-SOD levels can be calculated by comparing the absorbance compared to a standard curve constructed by measuring the activity of increasing and known amounts of Cu/Zn SOD (available from Sigma). Hemoglobin A_{1c} may be measured, for example, using high pressure (or performance) liquid chromatography (HPLC) and turbidimetric immunoinhibition methods (e.g. Synchron® available from Bechman Coulter)

Additionally, in various embodiments, the disease, disorder or condition is characterized in whole or in part by (a) hypercupremia and/or copper-related tissue damage and (b) one or more of hypertension, hyperlipidemia, impaired glucose tolerance, impaired fasting glucose, hyperglycemia, and insulin resistance, or predisposition to, or risk for, (a) and (b). In other embodiments, the disease, disorder or condition is selected from the group consisting of heart disease, glucose metabolism disorders, weight disorders, lipid disorders, and neurological disorders. Glucose metabolism include impaired glucose tolerance, impaired fasting glucose, prediabetes, type 1 diabetes, type 2 diabetes, insulin resistance, hyperglycemia, hyperinsulinemia, hyperamylinemia, and metabolic syndrome. Heart diseases include, for example, hypertension, atherosclerosis, heart failure, and cardiomyopathy. Weight disorders include, for example, obesity. Lipid disorders include, for example, hyperlipidemia, hypertriglyceridemia, and hypercholesterolemia. Neurological disorders include, for example, Alzheimer's disease, Huntington's Disease and Parkinson's disease. Subjects include mammals, for example, humans.

Also provided are methods for detecting the presence or risk of developing diabetic complications in a subject, the method comprising: correlating (i) a measurement of copper in a sample from the subject with (ii) a measure of glycemia, for example, a hemoglobin A_{1c} measurement for the subject and/or a measurement of extracellular superoxide dismutase or extracellular superoxide dismutase activity in a sample from the subject, wherein elevated copper and elevated hemoglobin A_{1c} and/or extracellular superoxide dismutase activity correlates with the presence of or risk of developing diabetic complications. In one embodiment, both hemoglobin A_{1c} and a measurement of extracellular superoxide dismutase are correlated with a copper measurement.

Also provided are methods for detecting the presence or risk of developing heart disease in a human, the method comprising: correlating (i) a measurement of copper in a sample from the subject with (ii) a measure of glycemia, for example, a hemoglobin A_{1c} measurement for the subject and/or a measurement of extracellular superoxide dismutase or extracellular superoxide dismutase activity in a sample from the subject, wherein elevated copper and elevated hemoglobin A_{1c} and/or extracellular superoxide dismutase activity correlates with the presence of or risk of developing heart disease. In one embodiment, both hemoglobin A_{1c} and a measurement of extracellular superoxide dismutase activity are correlated with a copper measurement.

Also provided are methods for detecting the presence or risk of developing neurological disorders, including Alzheimer's disease, Huntington's Disease, ALS, or Parkinson's disease in a subject, the method comprising, for example, correlating (i) a measurement of copper in a sample from the subject with (ii) a hemoglobin A_{1c} or other glycemic measurement for the subject and/or a measurement of extracellular superoxide dismutase or extracellular superoxide dismutase activity in a sample from the subject, wherein elevated copper and elevated hemoglobin A_{1c} and/or extracellular superoxide dismutase activity correlates with the presence of or risk of developing diabetic complications. In one embodiment, both hemoglobin A_{1c} and a measurement of extracellular superoxide dismutase activity are correlated with a copper measurement.

Also provided are methods for evaluating a compound for use in the treatment of a disease involving copper, the method comprising: a) administering the compound to a test subject for a predetermined period of time; b) obtaining one or more copper measurements from the test subject; c) obtaining one or more measurements of extracellular superoxide dismutase or extracellular superoxide dismutase activity (or superoxide) from the test subject; and d) correlating a change in copper and extracellular superoxide dismutase or extracellular superoxide dismutase activity (and/or superoxide) with effectiveness of the compound. In one embodiment, the method further comprises obtaining one or more glycemic measurements, e.g., one or more hemoglobin A_{1c} measurements. In another embodiment, extracellular superoxide dismutase activity is determined by measuring the amount of extracellular superoxide dismutase. In other embodiments, the disease, disorder or condition is selected from the group consisting of heart disease, glucose metabolism disorders, weight disorders, lipid disorders, and neurological disorders, as described above.

Also provide are methods of evaluating a subject for copper regulation therapy, which comprises obtaining at least one serum sample and/or at least one urine sample from the subject; obtaining, for example, a hemoglobin A_{1c} measurement from the subject; measuring copper concentration in a serum and/or urine sample from the subject; and, identifying the subject as a candidate for copper regulation therapy where the subject has, for example, (i) a hemoglobin A_{1c} of at least about 8% and (ii) a serum copper concentration of at least about 14 μM and/or a urine copper concentration of at least about 100 nM. In one embodiment, the subject has (i) serum copper of at least about 20 μM and (ii) for example, hemoglobin A_{1c} of at least about 6 to about 8%. In another embodiment, the method further comprises measuring extracellular superoxide dismutase activity in a serum (or plasma) sample from the subject, and identifying the subject as a candidate for copper regulation therapy where the subject has elevated extracellular superoxide dismutase activity. In other embodiments, extracellular superoxide dismutase activity is determined by measuring the amount of extracellular superoxide dismutase, and extracellular superoxide dismutase activity is at least about 1.5 times the upper limit of normal serum extracellular superoxide dismutase activity. In another embodiment, the method further comprises measuring superoxide in a subject, and identifying the subject as a candidate for copper regulation therapy where the subject has elevated superoxide. In other embodiments, the subject has a disease, disorder or condition selected from the group consisting of heart disease, glucose metabolism disorders, weight disorders, lipid disorders, and neurological disorders, as described above.

Also provided are methods for identifying a subject as a candidate for copper regulation therapy, which comprises determining in the subject levels of (i) copper and (ii) one or more of elevated hemoglobin A_{1c} and serum (or plasma) extracellular superoxide dismutase activity; and, identifying the subject as a candidate for copper regulation therapy based on elevated levels of (i) copper and (ii) hemoglobin A_{1c}, and/or serum or plasma extracellular superoxide dismutase activity. In other embodiments, extracellular superoxide dismutase activity is determined by measuring the amount of extracellular superoxide dismutase. In another embodiment, the subject has elevated copper, elevated hemoglobin A_{1c}, and elevated extracellular superoxide dismutase activity (and/or elevated superoxide). In one embodiment, elevated copper is determined by obtaining a serum sample from the subject and measuring serum copper. In other embodiments, elevated copper is determined by obtaining a urine sample from the subject and measuring urine copper. In other embodiments, the subject has a disease, disorder or condition selected from the group consisting of heart disease, glucose metabolism disorders, weight disorders, lipid disorders, and neurological disorders, as described above.

Also provided are methods for determining whether to initiate, continue, modify or terminate copper regulation therapy in a subject, which comprises measuring (a) copper and (b) one or more of hemoglobin A_{1c} and extracellular superoxide dismutase activity in the subject; and, determining whether to initiate, continue, modify or terminate copper regulation therapy for treatment of a disease, disorder or condition in the subject based on the measurement of (i) copper and (ii) one or more of hemoglobin A_{1c} and serum or plasma extracellular superoxide dismutase activity (and/or superoxide). In one embodiment, determination of whether to initiate, continue, modify or terminate copper regulation therapy for treatment of a disease, disorder or condition in the subject is based on the measurement of (i) copper, (ii) hemoglobin A_{1c} and (iii) extracellular superoxide dismutase activity (and/or superoxide). In another embodiment, the method further comprises the step of initiating or continuing copper regulation therapy in the subject when the subject is determined to have (a) elevated copper and (b) one or more of elevated hemoglobin A_{1c} and elevated extracellular superoxide dismutase activity (and/or superoxide). In another embodiment, the method further comprises modifying a copper regulation therapy regimen for the subject based on the measurement of (i) copper and (ii) one or more of hemoglobin A_{1c} and extracellular superoxide dismutase activity (and/or superoxide). In another embodiment, the method further comprises modifying a copper regulation therapy regimen for the subject when the subject is determined to have (a) elevated copper and (b) one or more of elevated hemoglobin A_{1c} and elevated extracellular superoxide dismutase activity (and/or superoxide) when compared to (i) at least one previous measurement of copper in the subject and (ii) at least one previous measurement of hemoglobin A_{1c} or extracellular superoxide dismutase activity (and/or superoxide) or both or all in the subject.

In another embodiment, the method further comprises modifying a copper regulation therapy regimen for the subject when the subject is determined to have (a) reduced levels of copper and (b) one or more reduced levels of hemoglobin A_{1c} and reduced extracellular superoxide dismutase activity when compared to (i) at least one previous measurement of copper in the subject and (ii) at least one previous measurement of hemoglobin A_{1c} or extracellular superoxide dismutase activity or both in the subject. In another embodiment, the method further comprises modifying a copper regulation therapy regimen to low dose copper antagonist therapy for the subject when the subject is determined to have (i) a serum copper concentration of less than about 14 μM and/or a urine copper concentration of less than about 100 nM; (ii) a hemoglobin A_{1c} of less than about 6 to less than about 8% and/or (iii) a extracellular superoxide dismutase activity of less than about 1.5 times the upper limit of normal. In yet another embodiment, the method further comprises the step of terminating copper regulation therapy in the subject when the subject is determined to have (i) a serum copper concentration of less than about 14 μM and/or a urine copper concentration of less than about 100 nM; (ii) a hemoglobin A_{1c} of less than about 6 to less than about 8% and/or (iii) a extracellular superoxide dismutase activity of less than about 1.5 times the upper limit of normal. In other embodiments, the copper is measured from serum, the serum copper is at least about 14 μM, the copper is measured from urine, the urine copper is at least about 100 nM per liter, the urine copper is at least about 300 nM per liter, and the urine copper is at least about 500 nM per liter. In still other embodiments, the extracellular superoxide dismutase activity is determined by measuring serum extracellular superoxide dismutase, and the extracellular superoxide dismutase is at least about 1.5 times the upper limit of normal. In yet other embodiments, the extracellular superoxide dismutase is at least about 40 Units per liter. In other embodiments, the subject has a disease, disorder or condition selected from the group consisting of heart disease, glucose metabolism disorders, weight disorders, lipid disorders, and neurological disorders, as described above.

In various embodiments of the methods, the copper regulation therapy comprises administering a copper antagonist and/or a pre-complexed copper antagonist. In various embodiments, the copper regulation therapy comprises administering a copper II antagonist. In still other embodiments, the copper regulation therapy comprises administering a copper H chelator. In yet other embodiments, the copper regulation therapy comprises administering a trientine-type compound, for example, triethylenetetramine dihydrochloride and/or triethylenetetramine disuccinate. In other embodiments, the copper regulation therapy comprises administering a copper II antagonist pre-complexed with a non-copper metal ion. In still other embodiments, the copper regulation therapy comprises administering a thiomolybdate. In other embodiments, the subject has a disease, disorder or condition selected from the group consisting of heart disease, glucose metabolism disorders, weight disorders, lipid disorders, and neurological disorders, as described above.

In various embodiments of the methods, the copper regulation therapy comprises administering a copper antagonist and/or a pre-complexed copper antagonist in combination with one or more of the following agents: an anti-obesity agent, a hypoglycemic agent, an anti-hypertension agent, an anti-diabetes agent, protein C or a protein C derivative, and a 3-hydroxy-3-methylglutaryl coenzyme A reductase inhibitor.

Anti-obesity agents may include, but are not limited to agents that lower body fat and include, for example, appetite suppressants, anorectics (including phentermine, mazindol, diethylpropion, and phendimetrazine), lipase inhibitors (including orlistat), exendins and exendin antagonists (including exendin-4), amylins and amylin agonists (including pramlinitide), leptins, GLP-1 and GLP-1 agonists (including Arg(34)Lys(26)-(N-ε-(γ-Glu(N-α-hexadecanoyl))-GLP-1(7-37), sometimes referred to herein as GLP-1LA)), and adrenergic receptor agonists (including sibutramine).

Suitable hypoglycemic agents may include, but are not limited to, biguanides (for example, metformin), thiazolidinediones (for example, troglitazone, rosiglitazone, and pioglitazone), α-glucosidase inhibitors (for example, acarbose and miglitol), and sulfonylureas (for example, tolbutamide, chlorpropamide, gliclazide, glibenclamide, glipizide, and glimepiride). Other hypoglycemic agents include amylin and amylin agonists (e.g., pramlintide, which is ^{25,28,29}Pro-h-amylin), GLP-1 and GLP-1 agonists (e.g., Arg(34)Lys(26)-(N-ε-(γ-Glu(N-α-hexadecanoyl))-GLP-1(7-37), or GLP-1LA)), and exendin and exendin agonists (e.g., exendin-4).

Suitable anti-hypertension agents are those that lower blood pressure and include, for example, diuretics (including hydrochloride and chlorthalidone), α-adrenergic receptor antagonists (including prazosin, terazosin, doxazosin, ketanserin, indoramin, urapidil, clonideine, guanabenz, guanfacine, guanadrel, reserpine, and metyrosine), β₁-selective adrenergic antagonist (including metoprolol, atenolol, esmolol, acebutolol, bopindolol, carteolol, oxprenolol, penbutolol, medroxalol, bucindolol, levobunolol, metipranolol, bisoprolol, nebivolol, betaxolol, celiprolol, sotalol, propafenone, propranolol, timolol maleate, and nadolol), ACE inhibitors (including captopriol, fentiapril, pivalopril, zofenopril, alacepril, enalapril, enalaprilat, enalaprilo, lisinopril, benazepril, quinapril, moexipril), calcium channel blockers (including nisoldipine, verapamil, diltiazem, nifedipine, nimodipine, felodipine, nicardipine, isradipine, amlodipine, and bepridil), angiotensin II receptor antagonists (including losartan, candesartan, irbesartan, valsartan, telmisartan, eprosartan, and olmesartan medoxomil), and vasodilators (including hydralazine, Minoxidil, sodium nitroprusside, diazoxide, bosentan, eporprostenol, treprostinil, and iloprost). Other antihypertensive agents include sympatholytic agents (e.g., methyldopa), ganglionic blocking agents (including mecamylamine and trimethaphan), and endothelin receptor antagonists (including bosentan and sitaxsentan).

Suitable anti-diabetic agents may include, but are not limited to insulin, amylin and amylin agonists, exendin and exendin angoinits, GLP-1 and GLP-1 agonists. Suitable insulins and insulin like compounds include (1) rapid-acting insulins (also sometimes referred to as “monomeric insulin analogs”); (2) short-acting insulins (also sometimes referred to as “regular” insulins); (3) intermediate-acting insulins; (4) long-acting (also sometimes referred to as “basal insulins”); (5) ultra-long acting insulins, (6) pI-shifted insulin analogs; (7) insulin deletion analogs; (8) derivatized insulins; (9) derivatized insulin analogs; (10) derivatized proinsulins; (11) human insulin analog complexes (e.g., hexamer complexes), (12) insulin mixtures, and (13) PEG-insulins.

Suitable biologically active protein C and protein C derivatives are those that increase anti-coagulation activity and may include, for example, isolated and/or purified protein C (e.g., protein C concentrate), recombinant protein C (e.g., drotrecogin alfa) and truncations or mutations thereof.

Suitable 3-hydroxy-3-methylglutaryl coenzyme A reductase inhibitors include the statins. Preferred statins are simvastatin, atorvastatin, lovastatin, pravastatin, fluvastatin, and rosuvastatin. Other statins include itavastatin and visastatin. Also provided are methods for qualifying a subject for copper regulation therapy, which comprises obtaining a hemoglobin A_{1c} measurement for the subject; obtaining a serum copper concentration measurement for the subject; and, identifying the subject as suitable for copper regulation therapy if the hemoglobin A_{1c} is at least about 8% and the serum copper is at least about 14 μM. In various embodiments, the serum copper is at least about 16 μM, at least about 18 μM, or at least about 20 μM. In other embodiments, the hemoglobin A_{1c} is at least about 6 to about 8%. In another embodiment, the method further comprises measuring serum extracellular superoxide dismutase activity, and identifying the subject as suitable for copper regulation therapy if (i) hemoglobin A_{1c} is at least about 8%, (ii) serum copper is at least about 14 μM, and (iii) the serum extracellular superoxide dismutase activity is at least about 1.5 times the upper limit of normal extracellular superoxide dismutase activity. In another embodiment, the method further comprises measuring serum extracellular superoxide dismutase activity, and identifying the subject as suitable for copper regulation therapy if (i) hemoglobin A_{1c} is at least about 8%, (ii) serum copper is at least about 14 μM, and (iii) the serum extracellular superoxide dismutase activity is at least about 40 Units per liter.

Also provided are methods for qualifying a subject for copper regulation therapy, which comprises obtaining a hemoglobin A_{1c} measurement for the subject; obtaining a urine copper measurement for the subject; and, identifying the subject as suitable for copper regulation therapy if the hemoglobin A_{1c} is at least about 8% and the urine copper is at least about 100 nM. In other embodiments, the urine copper is at least about 1.4 times the upper limit of normal urine copper, the urine copper is at least about 200 nM, the urine copper is at least about 300 nM, and the urine copper is at least about 500 nM. In still other embodiments, the method further comprises measuring serum extracellular superoxide dismutase activity, and identifying the subject as suitable for copper regulation therapy if (i) hemoglobin A_{1c} is at least about 8%, (ii) urine copper is at least about 100 nM, and (iii) the serum extracellular superoxide dismutase activity is at least about 1.5 times the upper limit of normal extracellular superoxide dismutase activity. In another embodiment, the method further comprises measuring serum extracellular superoxide dismutase activity, and identifying the subject as suitable for copper regulation therapy if (i) hemoglobin A_{1c}, is at least about 8%, (ii) urine copper is at least about 100 nM, and (iii) the serum extracellular superoxide dismutase activity is at least about 40 Units per liter.

In other embodiments of the methods, the methods further comprise obtaining one or more total cholesterol, LDL-cholesterol, VLDL-cholesterol, oxidized LDL-cholesterol, HDL-cholesterol, and/or triglyceride measurement(s) for the subject. In still other embodiments, the methods further comprise identifying the subject as suitable for copper regulation therapy if the total cholesterol is at least about 200 mg/dL, identifying the subject as suitable for copper regulation therapy if the LDL-cholesterol is at least about 130 mg/dL, identifying the subject as suitable for copper regulation therapy if the VLDL-cholesterol is at least about 30 mg/dL, identifying the subject as suitable for copper regulation therapy if the oxidized LDL-cholesterol is at least about 1.3 mg/dL, identifying the subject as suitable for copper regulation therapy if the HDL-cholesterol is less than about 35 mg/dL, identifying the subject as suitable for copper regulation therapy if the triglyceride is at least about 150 mg/dL, and identifying the subject as suitable for copper regulation therapy if the ratio of total cholesterol to HDL-cholesterol is greater than 6.4 and the subject is a man, or greater than 5.6 and the subject is a woman.

In still other embodiments of the methods, the methods further comprise obtaining one or more homocysteine and/or highly sensitive C-reactive protein measurement(s) for the subject. In other embodiments, the methods further comprise identifying the subject as suitable for copper regulation therapy if the homocysteine is at least about 11.4 μM/L and/or the highly sensitive C-reactive protein is at least about 1.0 mg/L.

Homocysteine levels are measured using methods known in the art. For example, such methods may include, but are not limited to, radio-enzymatic assay, ion-exchange chromatography, HPLC, GC-MS, and FPIA.

Also provided are methods for assessing the therapeutic effect of copper regulation therapy in a subject, comprising obtaining, for example, a serum sample from the subject; measuring hemoglobin A_{1c} and/or extracellular superoxide dismutase (or extracellular superoxide dismutase activity) in a serum sample from the subject sample; measuring serum copper concentration; and, comparing the hemoglobin A_{1c} and/or extracellular superoxide dismutase/extracellular superoxide dismutase activity and copper measurements with one or more previous hemoglobin A_{1c} and/or extracellular superoxide dismutase/extracellular superoxide dismutase activity and copper measurements from the subject and assessing the therapeutic effect. In other embodiments, extracellular superoxide dismutase (or extracellular superoxide dismutase activity) but not hemoglobin A_{1c} is measured. In other embodiments, both extracellular superoxide dismutase (or extracellular superoxide dismutase activity) and hemoglobin A_{1c} are measured. In other embodiments, superoxide (or a marker thereof) but not hemoglobin A_{1c} is measured. In other embodiments, both superoxide (or a marker thereof) and hemoglobin A_{1c} are measured. In still other embodiments, the method further comprises identifying the subject as suitable for copper regulation therapy if the homocysteine is measured at least about 11.4 μM/L.

Also provided are methods for assessing the therapeutic effect of copper regulation therapy in a subject, comprising obtaining a serum sample from the subject; measuring extracellular superoxide dismutase (or extracellular superoxide dismutase activity) in the serum sample; and, determining the effect of the copper regulation therapy on extracellular superoxide dismutase (and/or activity) in the subject. In another embodiment, the method further comprises measuring total serum copper or total urine copper or both in the subject. In another embodiment, the method further comprises measuring hemoglobin A_{1c} in the subject. In another embodiment, the method further comprises identifying the subject as suitable for copper regulation therapy if the homocysteine is at least about 11.4 μM/L.

Also provided are methods of treating a subject having (a) a serum copper level of at least about 14 μM and/or a urine copper level of at least about 100 nM, and (b) one or more of a hemoglobin A_{1c} of at least about 8% and a serum extracellular superoxide dismutase activity of at least about 40 Units per liter, comprising administering a therapeutically effective amount of a copper antagonist, for example, a copper (II) antagonist to the subject.

Also provided are methods of treating a subject for heart disease, the subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper antagonist, for example, a copper (II) antagonist to the subject. The heart disease may be selected from the group consisting of hypertension, atherosclerosis, heart failure, and cardiomyopathy. The atherosclerosis may include cerebrovascular atherosclerosis.

Also provided are methods of treating a subject for a glucose metabolism disorder, the subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper antagonist, for example, a copper (II) antagonist to the subject. The glucose metabolism disorder may be selected from the group consisting of impaired glucose tolerance, impaired fasting glucose, prediabetes, type 1 diabetes, type 2 diabetes, insulin resistance, hyperglycemia, hyperinsulinemia, hyperamylinemia, and metabolic syndrome.

Also provided are methods of treating a subject for a weight disorder, the subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper antagonist, for example, a copper (II) antagonist to the subject. Weight disorders include all classes of obesity.

Also provided are methods of treating a subject for a lipid disorder, the subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper antagonist, for example, a copper (II) antagonist to the subject. The lipid disorder may be selected from the group consisting of hyperlipidemia, hypertriglyceridemia, and hypercholesterolemia.

Also provided are methods of treating a subject for a neurological disorder, the subject having (a) a serum copper level of at least about 14 μM and/or a urine copper level of at least about 100 nM and (b) a serum extracellular superoxide dismutase activity of at least about 40 Units per liter, comprising administering a therapeutically effective amount of a copper antagonist, for example, a copper (II) antagonist to the subject. The neurological disorder may be selected from the group consisting of Alzheimer's disease, Huntington's Disease, ALS, and Parkinson's disease.

Also provided are methods of treatment, comprising administering a therapeutically effective amount of a copper antagonist, for example, a copper (II) antagonist to a subject with elevated oxidized LDL cholesterol. Also provided are methods of treating a subject with elevated oxidized LDL cholesterol, comprising administering a therapeutically effective amount of a copper antagonist, for example, a copper (II) antagonist to the subject. Also provided are methods of treating a subject with elevated oxidized LDL cholesterol, comprising administering a therapeutically effective amount of a copper antagonist, for example, a copper (II) antagonist to the subject.

In various embodiments, the methods further comprise means for the suppression of intravascular consumption of NO (nitric oxide). In still other embodiments, vascular superoxide production is lowered. In other embodiments, means for enhancing physiological vasodilatation is provided.

Assays capable of measuring chelatable copper are also provided, including assays for measuring chelatable copper in a sample comprising immobilizing a copper antagonist to a solid matrix; incubating said sample with said immobilized copper antagonist; rinsing non-specifically bound molecules from the solid matrix; eluting copper; and measuring copper levels using fluorescent spectrophotometery. In other embodiments the assay method further comprises an additional stringency step wherein a sample is incubated with a free ligand specific for non-copper metals. In other embodiments, the sample is a urine sample, a plasma sample, or a serum sample. Also provided are kits comprising an assay as provided herein with instructions for its use.

Also provided are methods of reducing serum or plasma EC-SOD levels or activity in a subject, comprising administering a therapeutically effective amount of a copper antagonist, for example, a copper (II) antagonist to said subject.

Also provided are methods of increasing tissue EC-SOD levels or activity, comprising administering a therapeutically effective amount of a copper antagonist, for example, a copper (II) antagonist to a subject. In some embodiments, said methods increase arterial or cardiovascular EC-SOD levels or activity in a subject.

Also provided are methods of increasing heparan sulfate levels in a subject, comprising administering a therapeutically effective amount of a copper antagonist, for example, a copper (II) antagonist to a subject.

Also provided are methods for treating a subject having a disorder characterized at least in part by elevated serum or plasma EC-SOD activity or mass, comprising administering to said subject a therapeutically effective amount of a copper (II) antagonist, whereby EC-SOD activity or mass is lowered.

Also provided are methods for treating a subject having a disorder characterized at least in part by decreased arterial or cardiovascular EC-SOD activity or expression, comprising administering to said subject a therapeutically effective amount of a copper (II) antagonist, whereby EC-SOD activity or expression is increased.

Also provided are treatment methods wherein a nitric oxide enhancer is administered together with a copper antagonist. Nitric oxide enhancers include, for example, nitrovasodilators, i.e., organic nitrates and nitrites and several other compounds that are capable of denitration to release nitric oxide (NO). Specific nitric oxide enhancers include, by way of example, nitroglycerin, isosorbide dinitrate, isosorbide-5-mononitrate, erythrityl tetranitrate, nitrosothiol S-nitroso-N-acetylpenicillamine, N-substituted piperazine NONOate compounds and other nitric oxide containing compounds (e.g., diazeniumdiolates, including O₂-aryl diazeniumdiolates), BiDil (NitroMed), and complexes of nitric oxide with polyamines (U.S. Pat. No. 5,155,137; U.S. Pat. No. 5,250,550). See also U.S. Pat. No. 5,366,997.

As noted above, pharmaceutically acceptable copper antagonists, preferably copper (II) antagonists, and more preferably copper (II) chleator agents, may be used in the invention. Copper antagonists include, for example, trientine active agents, which include trientines (triethylenetetramines).

Other suitable copper antagonists include, for example, pharmaceutically acceptable linear or branched tetramines capable of binding copper; 2,3,2 tetramine and salts thereof; 2,2,2 tetramine (also referred to as trientine) and salts thereof; 3,3,3 tetramine and salts thereof; triethylenetetramine hydrochloride salts, for example, triethylenetetramine dihydrochloride and triethylenetetramine tetrahydrochloride; triethylenetetramine succinate salts, for example, triethylenetetramine disuccinate; triethylenetetramine maleate salts, for example, triethylenetetramine tetramaleate and triethylenetetramine tetramaleate dihydrate; and triethylenetetramine fumarate salts, for example, triethylenetetramine tetrafumarate and triethylenetetramine tetrafumarate tetrahydrate.

Other suitable copper antagonists include, for example, crystalline triethylenetetramine and salts thereof. These include crystalline triethylenetetramine maleate (e.g., triethylenetetramine tetramaleate and triethylenetetramine tetramaleate dihydrate), crystalline triethylenetetramine fumarate (e.g., triethylenetetramine tetrafumarate and triethylenetetramine tetrafumarate tetrahydrate), and crystalline triethylenetetramine succinate (e.g., triethylenetetramine disuccinate anhydrate).

Useful agents may be prepared in a number of ways. For example, triethylenetetramine is a strongly basic moiety with multiple nitrogens that can be converted into a large number of suitable associated acid addition salts using an acid, for example, by reaction of stoichiometrically equivalent amounts of trientine and of the acid in an inert solvent such as ethanol or water and subsequent evaporation if the dosage form is best formulated from a dry salt. Possible acids for this reaction are in particular those that yield physiologically acceptable salts.

Nitrogen-containing copper antagonists, for example, trientine active agents such as, for example, trientine, that can be delivered as a salt(s) (such as acid addition salts, e.g., trientine dihydrochloride) act as copper-chelating agents or antagonists, which aids the elimination of copper from the body by forming a stable soluble complex that is readily excreted by the kidney. Thus inorganic acids can be used, e.g., sulfuric acid, nitric acid, hydrohalic acids such as hydrochloric acid or hydrobromic acid, phosphoric acids such as orthophosphoric acid, and sulfamic acid. This is not an exhaustive list. Other organic acids can be used to prepare suitable salt forms, in particular aliphatic, alicyclic, araliphatic, aromatic or heterocyclic mono- or polybasic carboxylic, sulfonic or sulfuric acids, (e.g., formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methanesulfonic acid, ethanesulfonic acid, ethanedisulfonic acid, 2-hydroxyethanesulfonic acid, benzenesulfonic acid, p-toluenesulfonic acid, naphthalenemono-and-disulfonic acids, and laurylsulfuric acid). Hydrochloric, maleic, fumaric, and succinic acid salts are preferred, and succinic acid salts are most preferred. Those in the art will be able to prepare these and other suitable salt forms.

Nitrogen-containing copper antagonists, for example, trientine active agents such as, for example, trientine, can also be in the form of quarternary ammonium salts in which the nitrogen atom carries a suitable organic group such as an alkyl, alkenyl, alkynyl or aralkyl moiety. In one embodiment such nitrogen-containing copper antagonists are in the form of a compound or buffered in solution and/or suspension to a near neutral pH much lower than the pH 14 of a solution of trientine itself.

Other trientine active agents include derivative trientines, for example, trientine in combination with picolinic acid (2-pyridinecarboxylic acid). These derivatives include, for example, trientine picolinate and salts of trientine picolinate, for example, trientine picolinate HCl. They also include, for example, trientine di-picolinate and salts of trientine di-picolinate, for example, trientine di-picolinate HCl. Picolinic acid moieties may be attached to trientine, for example one or more of the CH₂ moieties, using chemical techniques known in the art. Those in the art will be able to prepare other suitable derivatives, for example, trientine-PEG derivatives, which may be useful for particular dosage forms including oral dosage forms having increased bioavailability.

Other agents capable of reducing copper include those that decrease copper uptake, including thiomolybdates (including mono-, di-, tri- and tetrathiomolybdates); zinc salts, such as zinc acetate; zinc chloride; zinc sulfate; zinc salts of intermediates of the citric acid cycle, such as citrate, isocitrate, ketoglutarate, succinate, malate; and, zinc glucoante.

Copper antagonists useful in the invention also include copper antagonizing metabolites, such as copper antagonizing metabolites of trientine including, for example, N-acetyl trientine, and analogues, derivatives, and prodrugs thereof. Copper antagonists useful in the invention also include modified copper antagonists, for example, modified trientines. Derivatives of copper antagonists, including trientine or trientine salts or analogues, include those modified with polyethylene glycol (PEG).

Other copper antagonists include cyclic and acyclic compounds according to the formulae described in co-pending U.S. Published Patent Application No. 2006/0041170, filed Dec. 20, 2004 for “Copper Antagonist Compounds,” the contents of which are hereby incorporated by reference in its entirety (hereinafter “04 Copper Antagonist Compounds”).

Copper antagonists useful in the invention also include copper antagonists, that have been pre-complexed with a non-copper metal ion prior to administration for therapy. Metal ions used for pre-complexing have a lower association constant for the copper antagonist than that of copper. For example, a metal ion for pre-complexing a copper antagonist that chelates Cu²⁺ is one that has a lower binding affinity for the copper antagonist than Cu²⁺. Preferably, the non-copper metal ion has an association constant for triethylenetetramine that is equal to or less than about 10⁻¹⁹, more preferably less than or equal to about 10⁻¹⁸, still more preferably less than or equal to about 10⁻¹⁵, even more preferably less than or equal to about 10⁻¹², 10⁻¹⁰, or 10⁻⁹, and most preferably less than or equal to about 10⁻⁸, 10⁻⁷ or 10⁻⁵. Preferred metal ions for pre-complexing include, for example, calcium e.g., Ca²⁺), magnesium (e.g., Mg²⁺), chromium (e.g., Cr²⁺ and Cr³⁺), manganese (e.g., Mn²⁺), zinc (e.g., Zn²⁺), and iron (e.g., Fe²⁺ and Fe³⁺). Most preferred metal ions for pre-complexing are calcium, zinc, and iron. Other metals include, for example, cobalt (e.g., Co²⁺), nickel (e.g., Ni²⁺), silver (e.g., Ag¹⁺) and selenium (e.g., Se⁴⁺). Non-copper metals are chosen with regard, for example, to their relative binding to the copper antagonist, the dose of the copper antagonist to be administered, and relative to potential toxicity following displacement of the non-copper metal ion. In addition to free copper antagonist compounds and salts thereof, active metabolites, derivatives, and prodrugs of copper antagonists can also be used for pre-complexing. Preferred copper antagonists for pre-complexing are Cu²⁺ antagonists, particularly Cu²⁺ chelators. Preferred Cu²⁺ antagonists are linear, branched or cyclic polyamines chelators including, for example, tetramines. A preferred tetramine is triethylenetetramine. Examples of pre-complexed copper antagonists include pre-complexed triethylenetetramines. Pre-complexed triethylenetetramines include, for example, triethylenetetramine (or salts thereof, such as triethylenetetramine dihydrocholoride) pre-complexed with a metal ion having a binding constant lower than copper. Such compounds may be referred to, for example, as “Ca-Trientine” to refer to triethylenetetramine pre-complexed with calcium (e.g., Ca²⁺). Other copper antagonists include D-pencillamine, sar (N-methylglycine), diamsar (1,8-diamino-3,6,10,13,16,19-hexa-azabicyclo[6.6.6]icosane), N-acetylpenicillamine, N,N′-diethyldithiocarbamate, bathocuproinedisulfonic acid, bathocuprinedisulfonate, and thiomolybdates, including mono-, di-, tri- and tetrathiomolybdates. Each may be pre-complexed with a metal ion. Pre-complexed copper antagonists, for example, a pre-complexed triethylenetetramine, may be prepared as the pre-complexed compound or a salt thereof. Without intending to be bound to any particular mechanism or mode of action, pre-complexing is believed to assist in the preparation, stability, or bioavailability of copper antagonists, including those in to be prepared and administered in aqueous formulations, such as, for example, triethylenetetramine dihydrocholoride. This allows lower dosing as well. Pre-complexed copper antagonists may be present in the compositions of the invention in an amount, for example, that is effective to (1) increase copper output in the urine of said subject, (2) decrease body and/or tissue copper levels, and/or (3) decrease copper uptake, for example, in the gastrointestinal tract. Pre-complexed copper antagonists may be prepared and administered as described in U.S. Provisional Patent Application Ser. No. 60/665,234, filed Mar. 26, 2005 for “Pre-complexed Copper Antagonist Compounds.”

Also encompassed are metal complexes comprising copper antagonists and non-copper metals (that have lower binding affinities than copper for the copper antagonist) and one or more additional ligands than typically found in complexes of that metal. These additional ligands may serve to block sites of entry into the complex for water, oxygen, hydroxide, or other species that may undesirably complex with the metal ion and can cause degradation of the copper antagonist. For example, copper complexes of triethylenetetramine have been found to form pentacoordinate complexes with a tetracoordinated triethylenetetramine and a chloride ligand when crystallized from a salt solution rather than a tetracoordinate Cu²⁺ triethylenetetramine complex. In this regard, 219 mg of triethylenetetramine* 2 HCl were dissolved in 50 ml, and 170 mg of CuCl₂*2H₂O were dissolved in 25 ml ethanol (95%). After addition of the CuCl₂ solution to the triethylenetetramine solution, the color changed from light to dark blue and white crystals precipitated. The crystals were dissolved by addition of a solution of 80 mg NaOH in 15 ml H2O. After the solvent was evaporated, the residue was dissolved in ethanol, and two equivalents of ammonium-hexafluorophosphate were added. Blue crystals could be obtained after reduction of the solvent. Crystals were found that were suitable for x-ray structure determination. X-ray crystallography revealed a [Cu(triethylenetetramine)Cl] complex. Other coordinated complexes may be formed from or between copper antagonists, for example, copper chelators (such as Cu2+ chelators, spermadine, spermine, tetracyclam, etc.), particularly those subject to degradative pathways such as those noted above, by providing additional complexing agents (such as anions in solution, for example, I⁻, Br⁻, F⁻, (SO₄)²⁻, (CO₃)²⁻, BF⁴⁻, NO³⁻, ethylene, pyridine, etc.) in solutions of such complexes. This may be particularly desirable for complexes with more accessible metal ions, such as planar complexes or complexes having four or fewer coordinating agents, where one or more additional complexing agents could provide additional shielding to the metal from undesirable ligands that might otherwise access the metal and displace a desired complexing agent.

By way of example only, the dose amount of copper antagonist and/or pre-complexed copper antagonist, including, for example pre-complexed copper antagonists and pentacoordinate copper antagonist complexes, 04 Copper Antagonist Compounds and the like, preferably triethylenetetramine dihydrochloride and/or triethylenetetramine disuccinate and the like, may range from about 1 mg/kg to about 1 g/kg. Other therapeutically effective dose ranges include, for example, from about 1.5 mg/kg to about 950 mg/kg, about 2 mg/kg to about 900 mg/kg, about 3 mg/kg to about 850 mg/kg, about 4 mg/kg to about 800 mg/kg, about 5 mg/kg to about 750 mg/kg, about 5 mg/kg to about 700 mg/kg, about 5 mg/kg to about 600 mg/kg, about 5 mg/kg to about 500 mg/kg, about 10 mg/kg to about 400 mg/kg, about 10 mg/kg to about 300 mg/kg, about 10 mg/kg to about 200 mg/kg, about 10 mg/kg to about 250 mg/kg, about 10 mg/kg to about 200 mg/kg, about 10 mg/kg to about 200 mg/kg, about 10 mg/kg to about 150 mg/kg, about 10 mg/kg to about 100 mg/kg, about 10 mg/kg to about 75 mg/kg, about 10 mg/kg to about 50 mg/kg, or about 15 mg/kg to about 35 mg/kg.

In some embodiments of the invention, a therapeutically effective is from about 10 mg to about 4 g per day. Other therapeutically effective dose ranges include, for example, from about 20 mg to about 3.9 g, from about 30 mg to about 3.7 g, from about 40 mg to about 3.5 g, from about 50 mg to about 3 g, from about 60 mg to about 2.8 g, from about 70 mg to about 2.5 g, about 80 mg to about 2.3 g, about 100 mg to about 2 g, about 100 mg to about 1.5 g, about 200 mg to about 1400 mg, about 200 mg to about 1300 mg, about 200 mg to about 1200 mg, about 200 mg to about 1100 mg, about 200 mg to about 1000 mg, about 300 mg to about 900 mg, about 300 mg to about 800 mg, about 300 mg to about 700 mg or about 300 mg to about 600 mg per day.

In some embodiments of the invention, a therapeutically effective amount is an amount effective to elicit a plasma concentration of a copper antagonist, from about 0.01 mg/L to about 20 mg/L, about 0.01 mg/L to about 15 mg/L, about 0.1 mg/L to about 10 mg/L, about 0.5 mg/L to about 9 mg/L, about 1 mg/L to about 8 mg/L, about 2 mg/L to about 7 mg/L or about 3 mg/L to about 6 mg/L.

Other amounts may also be used. The amounts are not inflexible and may be determined, in part, for example, based on the number of tablets to be taken per day.

Additionally, copper antagonists including, for example pre-complexed copper antagonists and pentacoordinate copper antagonist complexes, 04 Copper Antagonist Compounds and the like, and the like, will be effective at doses in the order of 1/10, 1/50, 1/100, 1/200, 1/300, 1/400, 1/500 and even 1/1000 of those we have 1-50, 100, already employed (e.g., in the order of 120 mg.d⁻¹, 24 mg.d⁻¹, 12 mg.d⁻¹, etc.). The invention accordingly in part provides low dose compositions, formulations and devices comprising one or more copper antagonists (including precomplexed copper antagonists and pentacoordinate copper antagonist complexes) and/or one or more agents described herein. For example, low dose copper antagonists (including precomplexed copper antagonists and pentacoordinate copper antagonist complexes) may include compounds, including copper chelators, particularly Cu⁺² chelators, including but not limited to pre-complexed copper antagonists and pentacoordinate copper antagonist complexes, trientine active agents, including 04 Copper Antagonist Compounds, triethylenetetramine dihydrochloride and/or triethylenetetramine disuccinate, and the like, in an amount sufficient to provide, for example, dosages from 0.01 mg.kg⁻¹ to 5 mg.kg⁻¹, 0.01 mg.kg⁻¹ to 4.5 mg.kg⁻¹, 0.02 mg.kg⁻¹ to 4 mg.kg⁻¹, 0.02 to 3.5 mg.kg⁻¹, 0.02 mg.kg⁻¹ to 3 mg.kg⁻¹, 0.05 mg.kg⁻¹ to 2.5 mg.kg⁻¹, 0.05 mg.kg⁻¹ to 2 mg.kg⁻¹, 0.05-0.1 mg.kg⁻¹ to 5 mg.kg⁻¹, 0.05-0.1 mg.kg⁻¹ to 4 mg.kg⁻¹, 0.05-0.1 mg.kg⁻¹ to 3 mg.kg⁻¹, 0.05-0.1 mg.kg⁻¹ to 2 mg.kg⁻¹, 0.05-0.1 mg.kg⁻¹ to 1 mg.kg⁻¹, and/or any other doses or dose ranges within the ranges set forth herein. Low dose hypoglycemic agents, anti-obesity agents, statins, anti-hypertensive agents, protein C agents, may be included at doses in the order of 1/10, 1/50, 1/100, 1/200, 1/300, 1/400, 1/500 and 1/1000 of those doses used or described in the art.

Low dose combinations may comprise a low dose of copper antagonist(s) as described above and a regular dose of a hypoglycemic agent(s), anti-obesity agent(s), statin(s), anti-hypertensive agent(s), or protein C agent(s), as described herein; a regular dose of copper antagonist(s) as described herein and a lose dose of a hypoglycemic agent(s), anti-obesity agent(s), statin(s), anti-hypertensive agent(s), or protein C agent(s), as described above; and a low dose of copper antagonist(s) and a low dose of a hypoglycemic agent(s), anti-obesity agent(s), statin(s), anti-hypertensive agent(s), or protein C agent(s).

Among other things, low dose compositions of the invention may be used, for example, to prevent the onset of a disease, disorder or condition in a subject at risk of developing said disease, disorder or condition, or for maintenance therapy after a desired level of treatment has been reached to prevent the relapse or reoccurrence of a disease, disorder or condition. Any such dose may be administered by any of the routes or in any of the forms herein described. Aspects of the invention include controlled or other doses, dosage forms, formulations, compositions and/or devices containing one or more copper antagonists, wherein the copper antagonists are, for example, one or more 04 Copper Antagonist Compounds or trientine active agents, including but not limited to, trientine, trientine dihydrochloride or other pharmaceutically acceptable salts thereof, trientine analogues of 04 Copper Antagonist Compounds and salts thereof, and 04 Copper Antagonist Compounds pre-complexed with a non-copper metal ion. The present invention includes, for example, doses and dosage forms for at least oral administration, transdermal delivery, topical application, suppository delivery, transmucosal delivery, injection (including subcutaneous administration, subdermal administration, intramuscular administration, depot administration, and intravenous administration (including delivery via bolus, slow intravenous injection, and intravenous drip), infusion devices (including implantable infusion devices, both active and passive), administration by inhalation or insufflation, buccal administration, sublingual administration, and ophthalmic administration. It will be appreciated that any of the dosage forms, compositions, formulations or devices described herein particularly for oral administration may be utilized, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in an oral dosage form such as a modified release, extended release, delayed release, slow release or repeat action oral dosage form.

Any of the methods of treating a subject having or suspected of having or predisposed to a disease, disorder, and/or condition referenced or described herein may utilize the administration of any of the doses, dosage forms, formulations, compositions and/or devices herein described.

The invention may be carried out using doses, dosage forms, formulations, compositions and/or devices comprising one or more copper antagonists and/or pre-complexed copper antagonists, wherein the copper antagonist is, for example, one or more 04 Copper Antagonist Compounds, and salts thereof, including but not limited to, trientine, trientine dihydrochloride, trientine disuccinate, or other pharmaceutically acceptable salts thereof, or trientine analogues and salts thereof. The invention may be carried out, for example, using dosage forms, formulations, devices and/or compositions containing one or more copper antagonists and/or pre-complexed copper antagonists, wherein the copper antagonists are, for example, copper chelators, such as copper (II) chelators. The dosage forms, formulations, devices and/or compositions of the invention may be formulated to optimize bioavailability and to maintain plasma concentrations within the therapeutic range, including for extended periods. Controlled delivery preparations also optimize the drug concentration at the site of action and minimize periods of under and over medication, for example.

The dosage forms, devices and/or compositions useful in the invention may be provided for periodic administration, including once daily administration, for low dose controlled and/or low dose long-lasting in vivo release of a copper antagonist and/or a pre-complexed copper antagonist, wherein the copper antagonist is, for example, a copper chelator for chelation of copper and excretion of copper via the urine and/or to provide enhanced bioavailability of a copper antagonist, such as a copper chelator for chelation of copper and excretion of copper via the urine.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a copper antagonist and/or a pre-complexed copper antagonist.

Examples of dosage forms suitable for transdermal administration include, but are not limited, to transdermal patches, transdermal bandages, and the like. Examples of dosage forms suitable for topical administration of the compounds and formulations useful in the invention are any lotion, stick, spray, ointment, paste, cream, gel, etc., whether applied directly to the skin or via an intermed.

Examples of dosage forms suitable for suppository administration of the compounds and formulations useful in the invention include any solid dosage form inserted into a bodily orifice particularly those inserted rectally, vaginally and urethrally.

Examples of dosage forms suitable for transmucosal delivery of the compounds and formulations useful in the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations useful in the invention include delivery via bolus such as single or multiple administrations by intravenous injection, subcutaneous, subdermal, and intramuscular administration or oral administration.

Examples of dosage forms suitable for depot administration of the compounds and formulations useful in the invention include pellets or small cylinders of active agent or solid forms wherein the active agent is entrapped in a matrix of biodegradable polymers, microemulsions, liposomes or is microencapsulated.

Examples of infusion devices for compounds and formulations useful in the invention include infusion pumps containing one or more copper antagonists and/or pre-complexed copper antagonists, at a desired amount for a desired number of doses or steady state administration, and include implantable drug pumps.

Examples of implantable infusion devices for compounds and formulations useful in the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of compounds and formulations useful in the invention include compositions comprising solutions and/or suspensions in pharmaceutically acceptable, aqueous, or organic solvents, or mixture thereof and/or powders.

Examples of dosage forms suitable for buccal administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of dosage forms suitable for ophthalmic administration of the compounds and formulations useful in the invention include inserts and/or compositions comprising solutions and/or suspensions in pharmaceutically acceptable, aqueous, or organic solvents.

Examples of controlled drug formulations for delivery of the compounds and formulations useful in the invention are found in, for example, Sweetman, S.C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. Further guidance concerning the analysis of extended release dosage forms has been provided by the FDA See Guidance for Industry. Extended release oral dosage forms: development, evaluation, and application of in vitro/in vivo correlations. Rockville, Md.: Center for Drug Evaluation and Research, Food and Drug Administration (1997).

Further examples of dosage forms useful in the methods of the invention include, but are not limited to modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. U.S. Pharmacist 22 (Suppl.):3-12 (1997); Scale-up of oral extended-release drug delivery systems: part I, an overview, Pharmaceutical Manufacturing 2:23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. (1997) supra. Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

Also useful in the invention are coated beads, granules or microspheres containing one or more copper antagonists and/or pre-complexed copper antagonists, which may be used to achieve modified release of one or more copper antagonists and/or pre-complexed copper antagonists by incorporation of the drug into coated beads, granules, or microspheres. In such systems, the copper antagonist and/or pre-complexed copper antagonist is distributed onto beads, pellets, granules or other particulate systems. See Ansel, H. C., Allen, L. V. and Popovich, N. G., Pharmaceutical Dosage Forms and Drug Delivery Systems, 7th Ed., Lippincott 1999, p. 232.

Methods for manufacture of microspheres suitable for drug delivery have been described. See, for example, Arshady, R. Polymer Eng Sci 30:1746-1758 (1989); see also, Arshady, R., Polymer Eng Sci 30:905-914 (1990); see also: Arshady R., Polymer Eng Sci 30:915-924 (1990). Various coating systems are commercially available. E.g., Aquacoat [FMC Corporation, Philadelphia] and Surerelease™ [Colorcon]; Aquacoat aqueous polymeric dispersion. Philadelphia: FMC Corporation, 1991; Surerelease aqueous controlled release coating system. West Point, Pa.: Colorcon, 1990; Butler, J., et al., Pharm Tech 22:122-138 (1998); Yazici, E., et al., Pharmaceut Dev Technol 1:175-183 (1996).

Variation in the thickness of the coats and in the type of coating materials used affects the rate at which the body fluids are capable of penetrating the coating to dissolve the copper antagonist and/or a pre-complexed copper antagonist. Generally, the thicker the coat, the more resistant to penetration and the more delayed will be copper antagonist and/or a pre-complexed copper antagonist release and dissolution. See Madan, P. L. U.S. Pharmacist 15:39-50 (1990). This provides the different desired sustained or extended release rates and the targeting of the coated beads to the desired segments of the gastrointestinal tract. Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. (Each of Eudragit® RS and Eudragit® RL is an ammonio methacrylate copolymer. The release rate can be controlled not only by incorporating therein suitable water-soluble pore formers, such as lactose, mannitol, sorbitol, etc., but also by the thickness of the coating layer applied. Multi-tablets may be formulated which include small spheroid-shaped compressed mini-tablets that may have a diameter of between 3 to 4 mm and can be placed in a gelatin capsule shell to provide the desired pattern of copper antagonist and/or a pre-complexed copper antagonist release. Each capsule may contain 8-10 minitablets, some uncoated for immediate release and others coated for extended release of the copper antagonist and/or a pre-complexed copper antagonist.

A number of methods may be employed to generate modified-release dosage forms of one or more copper antagonists and/or a pre-complexed copper antagonist suitable for oral administration to humans and other mammals. Two basic mechanisms available to achieve modified release drug delivery include altered dissolution or diffusion of drugs and excipients. Within this context, for example, four processes may be employed, either simultaneously or consecutively. These are as follows: (i) hydration of the device (e.g., swelling of the matrix); (ii) diffusion of water into the device; (iii) controlled or delayed dissolution of the drug; and (iv) controlled or delayed diffusion of dissolved or solubilized drug out of the device. See, e.g., Examples 11, 12, 23, 24, 35, and 36 herein.

For orally administered dosage forms of the compounds and formulations of the invention, extended copper antagonist and/or pre-complexed copper antagonist action, for example, copper chelator action, may be achieved by affecting the rate at which the copper antagonist and/or pre-complexed antagonist is released from the dosage form and/or by slowing the transit time of the dosage form through the gastrointestinal tract (see Bogner, R. H., US Pharmacist 22 (Suppl.):3-12 (1997)). The rate of drug release from solid dosage forms may be modified by the technologies described below which, in general, are based on the following: 1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the copper antagonist is either coated or entrapped in a substance that is slowly digested or dispersed into the intestinal tract. The rate of availability of the copper antagonist and/or a pre-complexed copper antagonist is a function of the rate of digestion of the dispersible material. Therefore, the release rate, and thus the effectiveness of the copper antagonist and/or a pre-complexed copper antagonist, varies from subject to subject depending upon the ability of the subject to digest the material.

A further form of slow release dosage form of the compounds and formulations of the invention is any suitable osmotic system where semi-permeable membranes of for example cellulose acetate, cellulose acetate butyrate, cellulose acetate propionate, is used to control the release of copper antagonist and/or a pre-complexed copper antagonist. These can be coated with aqueous dispersions of enteric lacquers without changing release rate. An example of such an osmotic system is an osmotic pump device, such as the Oros™ device developed by Alza Inc. (U.S.A.).

Other devices useful in the methods of the invention utilize monolithic matrices including, for example, slowly eroding or hydrophilic polymer matrices, in which one or more copper antagonists and/or copper antagonists pre-complexed with a non-copper metal ion are compressed or embedded.

Monolithic matrix devices comprising compounds and formulations useful in the invention include those formed using, for example, copper antagonists dispersed in a soluble matrix, which become increasingly available as the matrix dissolves or swells; examples include hydrophilic colloid matrices, such as hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Copper antagonist and/or a pre-complexed copper antagonist release occurs as the polymer swells, forming a matrix layer that controls the diffusion of aqueous fluid into the core and thus the rate of diffusion of copper antagonist and/or a pre-complexed copper antagonist from the system.

In such systems, the rate of copper antagonist and/or a pre-complexed copper antagonist release depends upon the tortuous nature of the channels within the gel, and the viscosity of the entrapped fluid, such that different release kinetics can be achieved, for example, zero-order, or first-order combined with pulsatile release. Where such gels are not cross-linked, there is a weaker, non-permanent association between the polymer chains, which relies on secondary bonding. With such devices, high loading of the copper antagonist is achievable, and effective blending is frequent. Devices may contain 20-80% of copper antagonist and/or a pre-complexed copper antagonist (w/w), along with gel modifiers that can enhance copper antagonist diffusion; examples of such modifiers include sugars that can enhance the rate of hydration, ions that can influence the content of cross-links, and pH buffers that affect the level of polymer ionization. Hydrophilic matrix devices may also contain one or more pH buffers, surfactants, counter-ions, lubricants such as magnesium stearate (BP, USP) and a glidant such as colloidal silicon dioxide (USP; colloidal anhydrous silica, BP) in addition to copper antagonist and hydrophilic matrix.

Monolithic matrix devices comprising compounds and formulations useful in the invention also include those formed using, for example, copper antagonist and/or a pre-complexed copper antagonist particles are dissolved in an insoluble matrix, from which copper antagonist becomes available as solvent enters the matrix, often through channels, and dissolves the copper antagonist and/or a pre-complexed copper antagonist particles. Examples include systems formed with a lipid matrix, or insoluble polymer matrix, including preparations formed from Carnauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend of powdered components: lipids (20-40% hydrophobic solids w/w) which remain intact during the release process; copper antagonist and/or a pre-complexed copper antagonist, e.g., copper chelator; channeling agent, such as sodium chloride or sugars, which leaches from the formulation, forming aqueous micro-channels (capillaries) through which solvent enters, and through which copper antagonist and/or a pre-complexed copper antagonist is released. In this system, the copper antagonist and/or a pre-complexed copper antagonist is embedded in an inert insoluble polymer and is released by leaching of aqueous fluid, which diffuses into the core of the device through capillaries formed between particles, and from which the copper antagonist and/or a pre-complexed copper antagonist diffuses out of the device. The rate of release is controlled by the degree of compression, particle size, and the nature and relative content (w/w) of excipients. An example of such a device is that of Ferrous Gradumet (Martindale 33rd Ed., 1360.3). A further example of a suitable insoluble matrix is an inert plastic matrix. By this method, copper antagonists and/or a pre-complexed copper antagonist are granulated with an inert plastic material such as polyethylene, polyvinyl acetate, or polymethacrylate, and the granulated mixture is then compressed into tablets. Once ingested, the copper antagonist and/or a pre-complexed copper antagonist is slowly released from the inert plastic matrix by diffusion. See, for example, Bodmeier, R. & Paeratakul, O., J Pharm Sci 79:32-26 (1990); Laghoueg, N., et al., Int J Pharm 50:133-139 (1989); Buckton, G., et al., Int Pharm 74:153-158 (1991). The compression of the tablet creates the matrix or plastic form that retains its shape during the leaching of the copper antagonist and/or a pre-complexed copper antagonist and through its passage through the gastrointestinal tract. An immediate-release portion of copper antagonist and/or a pre-complexed copper antagonist may be compressed onto the surface of the tablet. The inert tablet matrix, expended of copper antagonist and/or a pre-complexed copper antagonist, is excreted with the feces. An example of a successful dosage form of this type is Gradumet (Abbott; see, for example, Ferro-Gradumet, Martindale 33rd Ed., p. 1860.4).

Further examples of monolithic matrix devices useful in the methods of the invention include compositions and formulations of the invention incorporated in pendent attachments to a polymer matrix. See, for example, Scholsky, K. M. and Fitch, R. M., J Controlled Release 3:87-108 (1986). In these devices, copper antagonists and/or a pre-complexed copper antagonist, e.g., copper chelators, may be attached by means of an ester linkage to poly(acrylate) ester latex particles prepared by aqueous emulsion polymerization. Still further examples of monolithic matrix devices of the invention incorporate dosage forms in which the copper antagonist and/or a pre-complexed copper antagonist is bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid. See Chafi, N., et al., Int J Pharm 67:265-274 (1992).

Two-layered tablets can be manufactured containing one or more of the compositions and formulations useful in the invention, with one layer containing an uncombined copper antagonist and/or a pre-complexed copper antagonist for immediate release and the other layer having a copper antagonist and/or a pre-complexed copper antagonist imbedded in a hydrophilic matrix for extended-release. Three-layered tablets may also be similarly prepared, with both outer layers containing the copper antagonist and/or a pre-complexed copper antagonist for immediate release. Some commercial tablets are prepared with an inner core containing the extended-release portion of drug and an outer shell enclosing the core and containing drug for immediate release. The invention may also be carried out using a copper antagonist and/or a pre-complexed copper antagonist complexed with an ion exchange resin, whereupon the complex may be tableted, encapsulated or suspended in an aqueous vehicle. Release of the copper antagonist and/or a pre-complexed copper antagonist is dependent on the local pH and electrolyte concentration such that the choice of ion exchange resin may be made so as to preferentially release the copper antagonist and/or a pre-complexed copper antagonist in a given region of the alimentary canal. Delivery devices incorporating such a complex may also be used, including, for example, a modified release dosage form.

Modified release forms of one or more copper antagonists and/or a pre-complexed copper antagonist, for example, one or more copper chelators and/or pre-complexed copper chelators, may also be prepared by microencapsulation. Microencapsulation is a process by which solids, liquids, or even gasses may be encapsulated into microscopic size particles through the formation of thin coatings of “wall” material around the substance being encapsulated such as disclosed in U.S. Pat. Nos. 3,488,418; 3,391,416 and 3,155,590. Gelatin (BP, USP) is commonly employed as a wall-forming material in microencapsulated preparations, but synthetic polymers such as polyvinyl alcohol (USP), ethylcellulose (BP, USP), polyvinyl chloride, and other materials may also be used. See, for example, Zentner, G. M., et al., J Controlled Release 2:217-229 (1985); Fites, A. L., et al., J Pharm Sci 59:610-613 (1970); Samuelov, Y., et al., J Pharm Sci 68:325-329 (1979). Different rates of copper antagonist and/or a pre-complexed copper antagonist release may be obtained by changing the core-to-wall ratio, the polymer used for the coating, or the method of microencapsulation. See, for example,: Yazici, E., Oner, et al., Pharmaceut Dev Technol; 1:175-183 (1996).

Other useful approaches include those in which the copper antagonist and/or a pre-complexed copper antagonist is incorporated into polymeric colloidal particles or microencapsulates (microparticles, microspheres or nanoparticles) in the form or reservoir and matrix devices. See: Douglas, S. J., et al., C.R. C. Crit. Rev Therap Drug Carrier Syst 3:233-261 (1987); Oppenheim, R. C., Int J Pharm 8:217-234 (1981); Higuchi, T., J Pharm Sci 52:1145-1149 (1963).

Also useful are repeat action tablets containing one or more copper antagonists and/or a pre-complexed copper antagonist, for example, one or more copper chelators. These are prepared so that an initial dose of the copper antagonist and/or a pre-complexed copper antagonist is released immediately followed later by a second dose. The tablets may be prepared with the immediate-release dose in the tablet's outer shell or coating with the second dose in the tablet's inner core, separated by a slowly permeable barrier coating. In general, the copper antagonist and/or a pre-complexed copper antagonist from the inner core is exposed to body fluids and released 4 to 6 hours after administration. Repeat action dosage forms are suitable for the administration of one or more copper antagonists and/or a pre-complexed copper antagonist for the indications noted herein.

Also useful are delayed-release oral dosage forms containing one or more copper antagonists and/or a pre-complexed copper antagonist, for example, one or more copper chelators and/or pre-complexed copper chelators. The release of one or more copper antagonists and/or a pre-complexed copper antagonist from an oral dosage form can be intentionally delayed until it reaches the intestine at least in part by way of, for example, enteric coating. Enteric coatings by themselves are not an efficient method for the delivery of copper antagonists and/or a pre-complexed copper antagonist because of the inability of such coating systems to provide or achieve a sustained therapeutic effect after release onset. Enteric coats are designed to dissolve or break down in an alkaline environment. Enteric coatings also have application when combined or incorporated with one or more of the other dose delivery formulations or devices described herein. The enteric coating may be time-dependent, pH-dependent where it breaks down in the less acidic environment of the intestine and erodes by moisture over time during gastrointestinal transit, or enzyme-dependent where it deteriorates due to the hydrolysis-catalyzing action of intestinal enzymes. See for example, Muhammad, N. A., et al., Drug Dev Ind Pharm., 17:2497-2509 (1991). Among the many agents used to enteric coat tablets and capsules known to those skilled in the art are fats including triglycerides, fatty acids, waxes, shellac, and cellulose acetate phthalate although further examples of enteric coated preparations can be found in the USP.

Also useful are devices incorporating one or more copper antagonists and/or a pre-complexed copper antagonist, for example, one or more copper chelators, in a membrane-control system. Such devices comprise a rate-controlling membrane enclosing a copper antagonist reservoir. Following oral administration the membrane gradually becomes permeable to aqueous fluids, but does not erode or swell. The copper antagonist and/or a pre-complexed copper antagonist reservoir may be composed of a conventional tablet, or a microparticle pellet containing multiple units that do not swell following contact with aqueous fluids. Active drugs) is/are released through a two-phase process, comprising diffusion of aqueous fluids into the matrix, followed by diffusion of the copper antagonist and/or a pre-complexed copper antagonist out of the matrix. Multiple-unit membrane-controlled systems typically comprise more than one discrete unit.

Yet further embodiments useful in the invention include formulations of one or more copper antagonists and/or a pre-complexed copper antagonist, for example, one or more copper chelators and/or pre-complexed copper chelators, incorporated into transdermal drug delivery systems, such as those described in: Transdermal Drug Delivery Systems, Chapter 10. In: Ansel, H. C., Allen, L. V. and Popovich, N. G. Pharmaceutical Dosage Forms and Drug Delivery Systems, 7th Ed., Lippincott 1999, pp. 263-278). Transdermal drug delivery systems facilitate the passage of therapeutic quantities of drug substances through the skin and into the systemic circulation to exert systemic effects, as originally described in Stoughton, R. D. Percutaneous absorption, Toxicol Appl Pharmacol 7:1-8 (1965). Evidence of percutaneous drug absorption may be found through measurable blood levels of the drug, detectable excretion of the drug and/or its metabolites in the urine, and through the clinical response of the subject to its administration.

Formulations of drugs suitable for transdermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion. See Shah, V., Peck, C.C., and Williams, R. L., Skin penetration enhancement: clinical pharmacological and regulatory considerations, In: Walters, K. A. and Hadgraft, J. (Eds.) Pharmaceutical skin penetration enhancement. New York: Dekker, (1993). Skin penetration enhancers suitable for formulation with copper antagonists in transdermal drug delivery systems may be chosen from the following list: acetone, laurocapram, dimethylacetamide, dimethylformamide, dimethylsulphoxide, ethanol, oleic acid, polyethylene glycol, propylene glycol and sodium lauryl sulfate. Further skin penetration enhancers may be found in publications known to those skilled in the art. See, for example, Osborne, D. W., & Henke, J. J., Pharm Tech 21:50-66 (1997); Rolf, D., “Pharm Tech 12:130-139 (1988). In addition to chemical means, there are physical methods that enhance transdermal drug delivery and penetration of the compounds and formulations of the invention. These include iontophoresis and sonophoresis. Formulations suitable for administration by iontophoresis or sonophoresis may be in the form of gels, creams, or lotions.

Transdermal delivery, methods or formulations of the invention, may utilize, among others, monolithic delivery systems, drug-impregnated adhesive delivery systems (e.g., the Latitude™ drug-in-adhesive system from 3M), active transport devices and membrane-controlled systems. Transdermal delivery dosage forms of the invention include those which substitute the copper antagonist, for the diclofenic or other pharmaceutically acceptable salt thereof referred to in the transdermal delivery systems disclosed in, by way of example, U.S. Pat. Nos. 6,193,996, and 6,262,121.

Formulations and/or compositions for topical administration of one or more compositions and formulations of the invention ingredient can be prepared as an admixture or other pharmaceutical formulation to be applied in a wide variety of ways including, but are not limited to, lotions, creams gels, sticks, sprays, ointments and pastes. These product types may comprise several types of formulations including, but not limited to solutions, emulsions, gels, solids, and liposomes. If the topical composition of the invention is formulated as an aerosol and applied to the skin as a spray-on, a propellant may be added to a solution composition. Suitable propellants as used in the art can be utilized. By way of example of topical administration of an active agent, reference is made to U.S. Pat. Nos. 5,602,125, 6,426,362 and 6,420,411.

Other dosage forms include variants of the oral dosage forms adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquify and/or dissolve in the rectal cavity to release the copper antagonist and/or a pre-complexed copper antagonist (e.g., copper chelator). Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7).

Transmucosal administration of the compounds and formulations useful in the invention may utilize any mucosal membrane but commonly utilizes the nasal, buccal, vaginal and rectal tissues. Formulations suitable for nasal administration of the compounds and formulations of the invention may be administered in a liquid form, for example, nasal spray, nasal drops, or by aerosol administration by nebulizer, including aqueous or oily solutions of the copper chelator and.or pre-complexed copper chelator. Formulations for nasal administration, wherein the carrier is a solid, include a coarse powder having a particle size, for example, of less than about 100 microns, preferably less, most preferably one or two times per day than about 50 microns, which is administered in the manner in which snuff is taken, i.e., by rapid inhalation through the nasal passage from a container of the powder held close up to the nose. Compositions in solution may be nebulized by the use of inert gases and such nebulized solutions may be breathed directly from the nebulizing device or the nebulizing device may be attached to a facemask, tent or intermittent copper antagonists may be administered orally or nasally from devices that deliver the formulation in an appropriate manner. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Extended rates of copper antagonist action following injection may be achieved in a number of ways, including crystal or amorphous copper antagonist and/or a pre-complexed copper antagonist forms having prolonged dissolution characteristics; slowly dissolving chemical complexes of the copper antagonist and/or a pre-complexed copper Antagonist formulation; solutions or suspensions of copper antagonist in slowly absorbed carriers or vehicles (as oleaginous); increased particle size of copper antagonist and/or a pre-complexed copper antagonist in suspension; or, by injection of slowly eroding microspheres of copper antagonist and/or a pre-complexed copper antagonist. See, e.g., Friess, W., et al., Pharmaceut Dev Technol 1:185-193 (1996).

Compositions may be prepared according to conventional methods by dissolving or suspending an amount of a copper antagonist(s) and/or a pre-complexed copper antagonist(s) ingredient in a diluent. The amount of copper antagonist and/or a pre-complexed copper antagonist is from between 0.1 mg to 1000 mg per ml of diluent. In some embodiments, dosage forms of 100 mg and 200 mg of a copper antagonist and/or a pre-complexed copper antagonist, for example, a copper chelator, are provided. By way of example only, the amount of copper antagonist and/or a pre-complexed copper antagonist, for example triethylenetetramine dihydrochloride or triethylenetetramine disuccinate may range from about 1 mg to about 750 mg or more (for example, about 1 mg, about 10 mg, about 25 mg, about 50 mg, about 100 mg, about 150 mg, about 200 mg, about 250 mg, about 400 mg, about 500 mg, about 600 mg, about 750 mg, about 800 mg, about 1000 mg, and about 1200 mg). Other amounts within these ranges may also be used and are specifically contemplated though each number in between is not expressly set out.

Copper antagonists and/or a pre-complexed copper antagonist can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations are isotonic or substantially isotonic to avoid irritation and pain at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to about 9.5 can enhance the stability of the parenteral formulation of the present invention. Other pH ranges, for example, include, about 5.5 to about 9.0, or about 6.0 to about 8.5, or about 6.5 to about 8.0, or, preferably, about 7.0 to about 7.5.

The buffer used may be selected from any of the following, for example, an acetate buffer, a phosphate buffer or glutamate buffer, the most preferred buffer being a phosphate buffer. Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents. A stabilizer may be included, but will generally not be needed. If included, however, an example of a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation. While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in “Remington's Pharmaceutical Sciences” as well as Pharmaceutical Dosage Forms: Parenteral Medications, Vol. 1, 1992, Avis et al. For these purposes, the copper antagonist and/or a pre-complexed copper antagonist may be administered parenterally (including subcutaneous injections, intravenous, intramuscular, intradermal injection or infusion techniques) or by inhalation spray in dosage unit formulations containing conventional non-toxic pharmaceutically acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant. It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

It is possible that other ingredients may be present in a parenteral pharmaceutical formulation useful the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention. Regarding pharmaceutical formulations, see also, Pharmaceutical Dosage Forms: Parenteral Medications, Vol. 1, 2nd ed., Avis et al., Eds., Mercel Dekker, New York, N.Y. 1992.

Suitable routes of parenteral administration include intramuscular, intravenous, subcutaneous, intraperitoneal, subdermal, intradermal, intraarticular, intrathecal and the like. Mucosal delivery is also permissible. The dose and dosage regimen will depend upon the weight and health of the subject.

In addition to the above means of achieving extended drug action, the rate and duration of copper antagonist and/or a pre-complexed copper antagonist delivery may be controlled by, for example by using mechanically controlled drug infusion pumps.

The copper antagonist(s) and/or a pre-complexed copper antagonist(s) can be administered in the form of a depot injection that may be formulated in such a manner as to permit a sustained release of the copper antagonist and/or a pre-complexed copper antagonist. The copper antagonist and/or a pre-complexed copper antagonist can be compressed into pellets or small cylinders and implanted subcutaneously or intramuscularly. The pellets or cylinders may additionally be coated with a suitable biodegradable polymer chosen so as to provide a desired release profile. The copper antagonist and/or a pre-complexed copper antagonist may alternatively be micropelleted. The copper antagonist and/or a pre-complexed copper antagonist micropellets using bioacceptable polymers can be designed to allow release rates to be manipulated to provide a desired release profile. Alternatively, injectable depot forms can be made by forming microencapsulated matrices of the copper antagonist and/or a pre-complexed copper antagonist in biodegradable polymers such as polylactide-polyglycolide. Depending on the ratio of copper antagonist and/or a pre-complexed copper antagonist to polymer, and the nature of the particular polymer employed, the rate of copper antagonist release can be controlled. Depot injectable formulations can also be prepared by entrapping the copper antagonist and/or a pre-complexed copper antagonist in liposomes, examples of which include unilamellar vesicles, large unilamellar vesicles and multilamellar vesicles. Liposomes can be formed from a variety of phospholipids, such as cholesterol, stearyl amine or phosphatidylcholines. Depot injectable formulations can also be prepared by entrapping the copper antagonist in microemulsions that are compatible with body tissue. By way of example, reference is made to U.S. Pat. Nos. 6,410,041 and 6,362,190.

Implantable infusion devices may employ inert material such as biodegradable polymers listed above or synthetic silicones, for example, cylastic, silicone rubber or other polymers manufactured by the Dow-Corning Corporation. The polymer may be loaded with copper antagonist and/or a pre-complexed copper antagonist and any excipients. Implantable infusion devices may also comprise a coating of, or a portion of, a medical device wherein the coating comprises the polymer loaded with copper antagonist and/or a pre-complexed copper antagonist and any excipient. Such an implantable infusion device may be prepared as disclosed in U.S. Pat. No. 6,309,380 by coating the device with an in vivo biocompatible and biodegradable or bioabsorbable or bioerodibleerodible liquid or gel solution containing a polymer with the solution comprising a desired dosage amount of copper antagonist and/or a pre-complexed copper antagonist and any excipients. The solution is converted to a film adhering to the medical device thereby forming the implantable copper antagonist-deliverable medical device. An implantable infusion device may also be prepared by the in situ formation of a copper antagonist and/or a pre-complexed copper antagonist containing solid matrix as disclosed in U.S. Pat. No. 6,120,789. Implantable infusion devices may be passive or active, as known in the art.

Delayed-release ocular preparations containing one or more copper antagonists and/or a pre-complexed copper antagonist, for example, one or more copper chelators, may be prepared with agents that increase the viscosity of solutions; by ophthalmic suspensions in which the copper antagonist particles slowly dissolve; by slowly dissipating ophthalmic ointments; or by use of ophthalmic inserts. Preparations of one or more copper antagonists and/or a pre-complexed copper antagonist suitable for ocular administration to humans may be formulated using synthetic high molecular weight cross-linked polymers such as those of acrylic acid (e.g., Carbopol 940) or gellan gum (Gelrite; see, Merck Index 12th Ed., 4389), a compound that forms a gel upon contact with the precorneal tear film (e.g. as employed in Timoptic-XE by Merck, Inc.). Further examples include delayed-release ocular preparations containing copper antagonist and/or a pre-complexed copper antagonist in ophthalmic inserts, such as the OCUSERT system (Alza Inc.). Typically, such inserts are elliptical with dimensions of about 13.4 mm by 5.4 mm by 0.3 mm (thickness).

Also useful are dose delivery formulations and devices formulated to enhance bioavailability of copper antagonist and/or a pre-complexed copper antagonist. This may be in addition to or in combination with any of the formulations or devices described above. Despite good hydrosolubility, one or more copper antagonists and/or a pre-complexed copper antagonist, for example, trientine, may be poorly absorbed in the digestive tract. By increasing the bioavailability of copper antagonists and/or a pre-complexed copper antagonist, a therapeutically effective level of a copper antagonist and/or a pre-complexed copper antagonist may be achieved by administering lower dosages than would otherwise be necessary. An increase in bioavailability of copper antagonists and/or a pre-complexed copper antagonist may be achieved by complexation of copper antagonists with one or more bioavailability or absorption enhancing agents or in bioavailability or absorption enhancing formulations. Such bioavailability or absorption enhancing agents include, but are not limited to, various surfactants such as various triglycerides, such as from butter oil, monoglycerides, such as of stearic acid and vegetable oils, esters thereof, esters of fatty acids, propylene glycol esters, the polysorbates, sodium lauryl sulfate, sorbitan esters, sodium sulfosuccinate, among other compounds.

Further examples of such agents include carrier molecules such as cyclodextrin and derivatives thereof, known in the art for their potential as complexation agents capable of altering the physicochemical attributes of drug molecules. For example, cyclodextrins may stabilize (both thermally and oxidatively), reduce the volatility of, and alter the solubility of, trientine active agents with which they are complexed. Cyclodextrins are cyclic molecules composed of glucopyranose ring units that form toroidal structures. The interior of the cyclodextrin molecule is hydrophobic and the exterior is hydrophilic, making the cyclodextrin molecule water-soluble. The degree of solubility can be altered through substitution of the hydroxyl groups on the exterior of the cyclodextrin. Similarly, the hydrophobicity of the interior can be altered through substitution, though generally the hydrophobic nature of the interior allows accommodation of relatively hydrophobic guests within the cavity. Accommodation of one molecule within another is known as complexation and the resulting product is referred to as an inclusion complex. Examples of cyclodextrin derivatives include sulfobutylcyclodextrin, maltosylcyclodextrin, hydroxypropylcyclodextrin, and salts thereof. Complexation of copper antagonist and/or a pre-complexed copper antagonist with a carrier molecule such as cyclodextrin to form an inclusion complex may thereby reduce the size of the copper antagonist and/or a pre-complexed copper antagonist dose needed for therapeutic efficacy by enhancing the bioavailability of the administered active agent.

Also useful in methods of the invention are microemulsions, i.e., such as fluid and stable homogeneous solutions composed of a hydrophilic phase, a lipophilic phase, at least one surfactant (SA) and at least one cosurfactant (CoSA). Examples of suitable surfactants include mono-, di- and triglycerides and polyethylene glycol (PEG) mono- and diesters. A cosurfactant, also sometimes known as “co-surface-active agentm,” is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

Copper antagonists and/or a pre-complexed copper antagonist may also be delivered using various polymers to enhance bioavailability by increasing adhesion to mucosal surfaces, by decreasing the rate of degradation by hydrolysis or enzymatic degradation of the copper antagonist and/or a pre-complexed copper antagonist, and by increasing the surface area of the copper antagonist and/or a pre-complexed copper antagonist relative to the size of the particle. Suitable polymers can be natural or synthetic, and can be biodegradable or non-biodegradable. Delivery of low molecular weight active agents, such as for example copper antagonists and/or a pre-complexed copper antagonist, may occur by either diffusion or degradation of the polymeric system. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Each of the polymers described above can be obtained from commercial sources such as Sigma Chemical Co., St. Louis, Mo., Polysciences, Warrenton, Pa., Aldrich Chemical Co., Milwaukee, Wis., Fluka, Ronkonkoma, N.Y., and BioRad, Richmond, Calif. or can be synthesized from monomers obtained from these suppliers using standard techniques.

The polymers described above can be separately characterized as biodegradable, non-biodegradable, and bioadhesive polymers. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of copper antagonists with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive copper antagonist and/or a pre-complexed copper antagonist (e.g., copper chelator and/or pre-complexed copper chelator) delivery systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and accordingly, these can also be used for bioadhesive copper chelator delivery systems.

Other agents that may enhance bioavailability or absorption of one or more copper antagonists can act by facilitating or inhibiting transport across the intestinal mucosa. For example, agents that increase blood flow, such as vasodilators, may increase the rate of absorption of orally administered copper antagonist and/or a pre-complexed copper antagonist by increasing the blood flow to the gastrointestinal tract. Vasodilators constitute another class of agents that may enhance the bioavailability of copper antagonists.

Other mechanisms of enhancing bioavailability of the compositions and formulations useful in the invention include the inhibition of reverse active transport mechanisms. For example, it is now thought that one of the active transport mechanisms present in the intestinal epithelial cells is p-glycoprotein transport mechanism which facilitates the reverse transport of substances, which have diffused or have been transported inside the epithelial cell, back into the lumen of the intestine. Inhibition of this p-glycoprotein mediated active transport system will cause less drug to be transported back into the lumen and will thus increase the net drug transport across the gut epithelium and will increase the amount of drug ultimately available in the blood. Various p-glycoprotein inhibitors are well known and appreciated in the art. These include, water soluble vitamin E; polyethylene glycol; poloxamers including Pluronic F-68; Polyethylene oxide; polyoxyethylene castor oil derivatives including Cremophor EL and Cremophor RH 40; Chrysin, (+)-Taxifolin; Naringenin; Diosmin; Quercetin; and the like.

A better understanding of the invention will be gained by reference to the following non-limiting experimental section which is illustrative and are not intended to limit the invention or the claims in any way.

### Example 1

Regulatory and human ethics approvals. Protocols incorporating experimental administration of triethylenetetramine dihydrochloride (trientine) to human subjects were approved in New Zealand by the Standing Committee on Therapeutic Trials (SCOTT), and by the Auckland Human Ethics Committee. All subjects provided written informed consent to participate.

Subjects. Male subjects aged 30-70 years with a normal ECG were recruited into this trial. Diabetic subjects were included if they were more than six months post type 2 diabetes diagnosis, and age-matched healthy control subjects were included on the basis of normal glucose tolerance established by standard oral glucose tolerance tests. Exclusion criteria included: confirmed diagnosis of T1DM; nephropathy (urine albumin>300 mg/1, [creatinine]ₛₑᵣᵤₘ>110 μM); abnormal hematology (hemoglobin<130 g/l, platelets<100×10⁹/l) or Fe deficiency anemia ([Fe]ₛₑᵣᵤₘ<10 μM, [ferritin]ₛₑᵣᵤₘ<20 μg/l); history of significant cardiac disease; previous hepatic, gastrointestinal or other endocrine disease; gangrene or active sepsis; severe retinopathy; non-diabetic renal disease or renal allograft; malignancy except cutaneous basal cell carcinoma; known abnormality of Cu or Fe metabolism; and current treatment with diuretics or Ca channel blockers.

Elemental balance studies. Males with diagnosed type 2 diabetes (n=20) and age-matched control subjects (n=20) underwent a factorial, randomized, double-blind, placebo-controlled elemental balance study performed at the University of Auckland Human Nutrition Unit. The study consisted of screening, enrollment, run-in, treatment and follow-up periods. All participants were required to be resident at the Human Nutrition Unit throughout the 12-day investigation period. Dietary trace metal intake was controlled and measured by providing all items of food and beverage consumed. Diets were constructed (Foodworks v2.10.136, Xyris Software, Brisbane, Australia, 2000) to adhere to the American Diabetes Association recommendations for diabetic patients, and included total fat of approximately 30% of total energy, saturated fatty acid intake of less than 10% of total energy, and cholesterol intake of less than 300 mg per day. Meal sizes were adjusted to each participant's body weight and activity levels to ensure energy balance, and subjects were requested to consume all foods provided. A three-day rotation of meals was used—two identical sets of meals were produced for each participant—one for consumption and the second for analysis to determine trace element content.

Rates of urinary excretion of Cu and other trace elements were determined from daily 24-hour urine collected during the 6-day baseline period. Fecal losses of trace elements were measured through the collection of all excreta on Days 1-6. Balances for Cu and other trace metals were calculated from the differences between amounts consumed in the diet and that excreted in the sum of urinary and fecal outputs. Fasting serum concentrations of trace elements were determined on the mornings of Days 1 and 7, the latter just before administration of drug. Upon completion of the baseline period (Days 1-6), subjects were randomized to placebo (methyl cellulose) or trientine (Anstead, U.K.; 2400 mg per day; 8 identical capsules to be taken at 8 am daily before breakfast, under supervision) and immediately entered the 6-day treatment period (Days 7-12). This was an identical regimen of dietary regulation and blood, urinary and fecal collection, which was completed on the morning of Day 13. There was no break between baseline and treatment periods.

Sample acquisition and analysis. Procedures to ensure accurate measurement of trace elements were performed throughout these studies. Serial collections of all 24-hour urines and of total daily fecal outputs were made into containers that were monitored by ICP-MS (see below) to verify freedom from elemental contamination; completeness of urine collection was estimated from recovery of parabenzoic acid (PABA). Fecal samples were freeze-dried and dry-weights recorded. Completeness of fecal collects was monitored in each subject by the introduction of opaque markers into the food on the morning of Day 7 and verification of recovery by X-ray analysis of freeze-dried fecal samples and counting of markers, whose mean recovery was 93-97% for all subjects. Total balance of Cu, Fe, Zn, Ca, Mg, Mn, Mo, Se and Cr were determined in a GLP-certified laboratory by ICP-MS in aliquots of 24-hour urines, and acid (HNO₃, Aristar) extracts of freeze-dried feces. Vanadium was not included in the analysis as preliminary studies indicated that its concentrations were below the m.d.c. for available methods, and would have been uninformative.

As expected, fecal frequency varied significantly between subjects, in some of whom it was ≦once per three-days. Therefore, we combined samples into 6-day balances for analysis, so as to minimise the effects of variable fecal frequency on total balance.

Biochemical and hematological variables were measured in fasting blood samples collected at Days 1 and 7 (before drug) and Day 13 (after drug): these consisted of serum Cu, Zn, Ca, Mg, Fe studies (Fe, IBC, ferritin), EC-SOD, liver function tests (bilirubin, aspartate aminotransferase, alanine amino transferase, alkaline phosphatase, γ-glutamyl-transferase and albumin), total protein, lipids (total cholesterol, high-density lipoprotein cholesterol, low-density lipoprotein cholesterol and triglycerides), glucose, HbA_{1c}, creatinine and creatinine clearance, and a full blood count. Serum concentrations of Mn, Mo, Se, and Cr were also determined on these days. In addition, 2-hour [Mg]ₛₑᵣᵤₘ was measured for 10 hours post-dose to exclude an effect of drug.

### Example 2

Dose-dependent effects of trientine on urinary metal excretion. We also performed a substudy to characterise dose-dependent effects of trientine on urinary excretion of Cu, Fe, Zn and the six other trace elements in people with or without type 2 diabetes, at and below the dose-range (1200 to 2400 mg/day) previously recommended for treatment of patients with Wilson's disease. Increasing doses of trientine (in mg/day: 300, 600, 1200 and 2400) were successively administered in an unblinded study for 1-week periods with 6-weeks washout between each dose. Blood and urine samples were collected before and after each 1-week treatment period. Subjects were 7 non-diabetic controls and 7 patients with type 2 diabetes who had completed the elemental balance study and agreed to participate in the substudy. A history, physical examination and safety laboratory tests (as above) were performed prior to each medication cycle, to confirm that the participants continued to meet all inclusion/exclusion criteria. Two weeks after each cycle, the participants were contacted by telephone to confirm their wellbeing and to check for any adverse events. To allow adequate washout and re-equilibration of blood and tissue trace metal concentrations, no subject was entered into the substudy until at least 6 weeks after completion of the main elemental balance study. The total substudy duration was (1+6) weeks×4 doses=28 weeks.

Elemental analysis. Elemental concentrations were measured in urine, serum, feces and food, by ICP-MS (Perkin Elmer-Sciex Elan 6100 DRC plus) according to optimized protocols with gallium as the internal standard, as recommended for elements measured with the instrument in Dynamic Reaction Cell mode with NH₃ as the reaction gas. Operating parameters were as follows: RF power, 1500 W; nebulizer gas flow rate, 0.9 l/min; auxiliary gas flow rate, 1.2 l/min; plasma gas flow rate, 15 l/min; reaction gas, NH₃ at 0.3 l/min; data acquisition mode, peak hopping, 3 replicates, 20 sweeps per replicate; sample uptake rate 1 l/min. Calibration standards were matrix-matched to samples: for example, for samples in 1% HNO₃ (v/v) such as diluted urine and serum, calibration standards were constituted in 1% HNO₃, whereas for fecal digests, standards were constituted in 5% HNO₃. Samples were analyzed in batches of 40-50; for each batch, a calibration-verification standard was measured, with standard elements derived from a source distinct from those employed in the calibration standards. Calibration standards were also included at every 20th position within assays to correct for within-run instrumental drift.

Statistical methods. Statistical analyses were performed using JMP 5.1 (SAS Institute, 2003), S-PLUS v6.1 (Insightful Corporation, Seattle, Wash., 2002) and SPSS v12.0.1 (SPSS Inc, Chicago, Ill., 2003). Baseline levels of variables from healthy (control) and diabetic patients were compared using one-way analysis of variance (ANOVA). RIGLS models for relationships between EC-SOD, [Cu]ₛₑᵣᵤₘ and HbA₁ₐ were fitted by restricted maximum likelihood (REML). Effects of subject status (control or diabetic) and treatment (placebo or trientine) were analyzed using a factorial experimental design with two time periods: Days 0 to 6 and Days 7 to 12, respectively. No drugs were administered to subjects regardless of treatment or subject status during the first period, so the interaction effect is the term of particular interest in this analysis. Differences between time periods for each subject were used in a mixed-model analysis of variance fitted by REML in which subjects were considered to be random factors. ANOVA was performed to test for differences between the treatments (trientine or placebo), subject status (diabetic or control) and the interaction between treatment and subject status. Responses included balance of 9 elements, and their urinary and fecal excretion, each of which was tested separately. Assumptions of normality and homoscedasticity were directly tested, and residuals were shown to be normally distributed. In a few cases, elemental concentrations were below detectable concentrations; these are indicated in tables where appropriate. Means and 95% CI are reported for all variables. Pearson's correlation coefficients were calculated to determine the relationship between baseline Cu and key variables. A model predicting urinary Cu excretion during Days 7-12 was constructed by addition of predictive baseline (Day 1) variables into a forward stepwise multiple regression model (with [symbol] =0.25 for a term entering the model and [symbol] =0.10 for a term leaving the model) to determine which variables were associated with baseline urinary Cu excretion; the coefficients for each parameter are reported for the final model. A significance level of [symbol] =0.05 was used for all statistical tests.

Baseline group characteristics. One control subject (trientine-treated group) withdrew during the treatment phase, leaving 20 type 2 diabetes completers and 19 control completers. Age, BMI and relevant blood analytes and hematological indices for the two study groups at baseline are presented in Table 1. Subjects with diabetes had comparable ages, but greater BMI, fasting plasma glucose, and HbA_{1c} values than controls (all P<0.001). Groups were well matched for serum concentrations of Cu, Fe, Zn, Ca, Mn and Se but, consistent with previous reports, [Mg]ₛₑᵣᵤₘ was lower (P<0.01) and [ferritin]ₛₑᵣᵤₘ was elevated (P<0.001) in diabetic subjects compared with controls, whereas IBC values were equivalent.

Relationship between serum HbA_{1c} and the interaction between EC-SOD and [Cu]ₛₑᵣᵤₘ. Serum EC-SOD was elevated at baseline (Day 1, Table 1) and on Day 7 (both P<0.01) in diabetic subjects, in whom it was related to [Cu]ₛₑᵣᵤₘ (EC-SOD=19.6.[Cu]ₛₑᵣᵤₘ−228; r²=0.16, P<0.05), whereas no equivalent relationship was present in control subjects. Contrastingly, baseline serum EC-SOD did not significantly correlate with any other [element]ₛₑᵣᵤₘ in diabetic subjects in univariate analysis. EC-SOD was related to an interaction between [Cu]ₛₑᵣᵤₘ and HbA_{1c} in RIGLS models on both Day 1 ([Cu]ₛₑᵣᵤₘ and HbA_{1c}, each P<0.05; interaction term, P<0.01) and Day 7 ([Cu]ₛₑᵣᵤₘ, HbA_{1c} and interaction term, all P<0.0001). A 3-D spline-surface illustrating the relationship on Day 7 is shown in FIG. 1. On Day 13, by contrast, following 6-days' trientine treatment, serum EC-SOD was significantly lower (31.8 U/l (6.9-56.6)) than on Days 1 and 7 (both P<0.05). Furthermore, EC-SOD was not significantly related to interactions between serum concentrations of any other element and HbA_{1c} (RIGLS: all P=ns), nor was HbA_{1c}, alone significantly related to EC-SOD in either diabetic or control subjects. In summary, the relationship between EC-SOD and [Cu]ₛₑᵣᵤₘ was markedly strengthened by inclusion of its interaction with HbA_{1c} in the RIGLS models, and 6-days' treatment with trientine suppressed the elevation of EC-SOD.

Serum ferritin. Consistent with previous reports, [ferritin]ₛₑᵣᵤₘ was elevated in diabetic subjects compared with controls (Table 1), but its values did not correlate significantly with [Fe]ₛₑᵣᵤₘ, IBC, [hemoglobin]_{blood} or packed cell volume (PCV) in diabetic subjects (RIGLS, all P=ns). These findings are consistent with previous reports, which have indicated that elevated ferritin may not be related to alterations in Fe homeostasis in type 2 diabetes (see discussion).

Baseline elemental balance. Baseline balance of nine elements in control or diabetic subjects, and corresponding urinary and fecal excretion rates, are presented in Table 2. Measured food intakes did not differ between groups or as a result of treatment, so elemental intakes have not been presented in Tables 2 and 4, although actual measured elemental intakes were employed in the balance calculations. Balance did not differ significantly between control and diabetic groups for any element studied. Cu balance was highly variable between subjects but tended to be more positive in diabetic than control subjects, although the difference was not significant (Table 2, P=0.18). Urinary excretion rates for Cu, Fe, Zn, Ca, Mn, Se and Cr were significantly higher in diabetes, and baseline urinary Cu excretion was closely correlated with that of Fe (univariate least squares regression: [Fe]_{24 h urine}=1.94.[Cu]_{24 h urine}+0.53; r²=0.48, P<0.0001). Thus, increased basal urinary Cu excretion in diabetes is closely related to increases in urinary excretion of Fe.

Effects of drug treatment on elemental balance. Effects of drug treatment (Placebo/Trientine) and of interactions between trientine and metabolic status (Control/Diabetes) on elemental balance were examined by fitting ANOVA models by REML to the balance, and the urinary and fecal excretion of each element. Resulting P values are shown in Table 3, and effects of drug treatment on differences between control and diabetic subjects on balance, and urinary and fecal excretion of Cu, Fe, Zn, Mn and Ca, are shown in Table 4.

Copper—Trientine treatment modified Cu balance in the whole study group (Table 3, P=0.0224) and the interaction term was significant (Table 3, P=0.0028). Trientine decreased Cu-balance in diabetic subjects compared with placebo (Table 4, P<0.001) but, by contrast, was without effect on balance in control subjects (Table 4, P=ns). Trientine treatment influenced urinary Cu excretion (Table 3, P<0.001) whereas the urinary trientine-diabetes interaction term was of borderline significance (Table 3, P=0.0748). Trientine stimulated urinary Cu excretion in both diabetic and control subjects (Table 4, P<0.001 in each group), indicating that it can extract Cu(II) from the body in both groups. In addition, the interaction term was significant for fecal Cu excretion (Table 3, P=0.0034); trientine's effect on fecal Cu was evoked mainly through suppression of fecal Cu excretion in controls (Table 4, P<0.001) whereas by contrast it was without significant effect on fecal Cu excretion in diabetic subjects (Table 4). These observations are consistent with trientine-mediated enhancement of Cu absorption from the gut in control subjects, and point to significant differences in the actions of trientine between diabetic and control subjects.

Iron—Trientine treatment also significantly affected Fe balance in the whole study group (Table 3, P=0.0278) but the corresponding trientine-metabolic status interaction term was not significant (Table 4). Trientine treatment increased Fe balance in control (Table 4, P<0.05) but not diabetic subjects. Basal urinary Fe was also elevated in diabetic compared to control subjects (Table 2, P<0.001); this elevation remained during, but was unaffected by trientine treatment, which was without effect on urinary Fe excretion in either diabetic or control subjects (Table 4). Fecal Fe excretion in the whole group was modified by trientine treatment (Table 3, P=0.0187) but the corresponding trientine-metabolic status interaction term was not significant. Trientine treatment significantly lowered fecal Fe excretion in control subjects (Table 4, P<0.05); although a similar trend was present in diabetic subjects, it was not significant (Table 4, P=ns). These data are consistent with trientine-mediated increases in Fe absorption from the gut in control subjects.

Zinc—Trientine treatment exerted a significant effect on Zn balance (Table 3, P=0.0021), and the corresponding trientine-metabolic status interaction term was also significant (P=0.002). Pretreatment (Day 1) urinary Zn was significantly elevated in diabetes (Table 2, P<0.001). Trientine treatment influenced urinary Zn excretion in the whole group (Table 3, P<0.0001) via stimulation in both control (Table 4, P<0.001) and diabetic (P<0.001) subjects. Trientine treatment also modified fecal Zn excretion in all subjects (Table 3, P<0.0001) and the trientine-metabolic status interaction term was also significant (P=0.0045). Trientine treatment decreased fecal Zn excretion in control subjects (Table 4, P<0.001) but, although a similar trend was present in diabetic subjects, it was not significant. These data indicate that trientine elicits increased Zn absorption from the gut in non-diabetic subjects, and trientine modified Zn balance in control subjects both by stimulating urinary Zn excretion and by stimulating Zn absorption. By contrast, detectable effects in diabetic subjects were limited to stimulation of urinary Zn excretion, although there was a similar but non-significant trend on fecal Zn absorption.

Calcium—Trientine treatment significantly modified Ca balance in the whole group (Table 3, P=0.0022) as the result of an increase Ca balance in controls (Table 4, P<0.01), but did not influence urinary Ca excretion. Interactions terms between drug and metabolic status were significant for both Ca balance (Table 3, P=0.0333) and fecal Ca excretion (Table 3, P=0.0204). Trientine strongly modified fecal Ca excretion (Table 3, P=0.0014) mainly through its suppression in control subjects (Table 4, P<0.01). The effects of trientine on Ca balance via decreased fecal Ca excretion in control subjects is consistent with drug-mediated stimulation of systemic uptake Ca from the gut. In diabetes, by contrast, trientine was without significant effects on Ca balance, or urinary or fecal Ca excretion, although non-significant trends were apparent. In this study, Ca metabolism in diabetic subjects was thus more resistant to the effects of trientine than that in corresponding controls.

Magnesium—Although basal [Mg]ₛₑᵣᵤₘ was elevated in control compared with diabetic subjects (Table 1), indices of basal Mg balance did not differ between diabetic and control subjects, nor were they modified by trientine treatment (data not shown).

Manganese—Trientine evoked significant effects on Mn balance (Table 3, P=0.0283) and fecal Mn excretion (Table 3, P=0.0172) in the whole group. Trientine-metabolic status terms were significant both for Mn balance (Table 3, P=0.0430) and fecal Mn excretion (Table 3, P=0.0342), indicating that trientine-modified Mn balance in the whole group occurred mainly as a result of fecal excretion. Effects of trientine on Mn balance were similar to those on Ca, evoked mainly via decreased fecal Mn excretion in control subjects and consistent with drug-mediated stimulation of systemic uptake Mn from the gut (results not shown).

Selenium—Trientine-metabolic status interaction terms were also significant for Se balance (Table 3, P=0.0157) and for fecal Se excretion (Table 3, P=0.0125). Effects of trientine on Se balance were similar to those for Ca and Mn, being evoked mainly via decreased fecal Se excretion in control subjects (results not shown).

Molybdenum/Chromium—Neither diabetes nor trientine-treatment affected balance of Mo or Cr, or their urinary or fecal excretion rates (results not shown).

Dose-dependent effects of trientine on metal excretion. Trientine caused dose-dependent increases in urinary Cu output in both diabetic ([Cu]_{24 hour urine}=0.00245.[trientine dose]+0.192, r²=0.66, P<0.0001) and non-diabetic ([Cu]_{24 hour urine}=0.00183.[trientine dose]+0.177, r²=0.56, P<0.0001) subjects, and gradients for Cu-excretion were not significantly different between groups. Trientine also dose-dependently stimulated urinary Zn excretion in both diabetic ([Zn]_{24 hour urine}==0.0158. [trientine dose]+14.5, r²=0.54, P<0.0001) and non-diabetic ([Zn]_{24 hour urine}=0.00967. [trientine dose]+4.32, r²=0.42, P<0.0001) subjects; however, in this case, the gradient in diabetic subjects was significantly greater than in controls (P<0.05). Dose-dependent increases in urinary Cu and Zn excretion are consistent with a previous report in which trientine was administered to healthy human subjects. By contrast, trientine had no dose-dependent effects on 24-hour urinary excretion of Fe or any other trace elements that were evaluated (results not shown).

Baseline variables that predict urinary Cu excretion during Days 7 to 12. A multivariate regression model was calculated which relates baseline variables to urinary Cu excretion during Days 7 to 12 (summarized in Table 5). Trientine treatment and diabetes were positive factors in this model. Urinary Cu excretion during Days 1 to 6 and baseline [Mg]ₛₑᵣᵤₘ were continuous variables that were significant positive components in the model, whereas baseline [ferritin]ₛₑᵣᵤₘ was a negative component.

### Example 3

This Example describes the measurement of free copper in serum and urine. Trace metal cleaned plastic ware (polyethylene, polypropylene, or Teflon) is used for all steps of the experiments: bottles are soaked in 10% HCl for >24 h, rinsed with ultrapure water (Barnstead NANOpure Diamond, Dubuque, Iowa), stored filled with 0.1% HCl, and then rinsed again with ultrapure water before use. To minimize the potential for Cu contamination, most of the sample handling is conducted on a Class 100 clean bench.

The gellyfish sampler (GFbeads) consists of iminodiacetate cation-exchange resin beads (Toyopearl AF-Chelate 650M, TosoHaas Biosep LLC; Montgomeryville, Pa.) embedded in a polyacrylamide gel matrix (modified from ref 29). The resin beads (hereafter referred to as beads) are shipped suspended in 20% methanol, with a mean bead size of 65 μm. The concentration of iminodiacetate groups is approximately 18 μmol per ml of resin slurry. Because of rapid gelling of the matrix, 5 mL batches of gel are prepared, each of which made approximately 16 gellyfish. Batches consist of 2.38 mL of ultrapure water, 0.75 mL of DGT gel cross-linker (2%; DGT Research Ltd.; Lancaster, UK), 1.88 mL of acrylamide solution (40%), 40 μl, of ammonium persulfate (10%, prepared within 24 h), 15 μL of TEMED(N,N,N′N′-tetramethylethylenediamine, 99%), and 100 μL of Tosohaas resin beads. Prior to addition to the gel solution, the beads are washed 3 times with ultrapure water to remove the liquid carrier. TEMED and ammonium persulfate quantities are adjusted to yield an optimal gel coagulation rate that allows sufficient time for mixing and pipetting yet minimizes bead settling prior to coagulation. The batch is repeatedly mixed, and 300 μL is pipetted into 16 polypropylene custom drilled molds. After the gelfully set (approximately 30 min), the GFbeads are transferred to a Teflon beaker containing ultrapure water and are rinsed and resuspended into clean water at least 3 times over the course of 24 h. GFbeads are stored in ultrapure water for up to 2 weeks before use and are rinsed periodically during that time. Fully hydrated gellyfish had dimensions of diameter approximately 2 cm and thickness approximately 2 mm and were >95% water with a wet volume of approximately 0.65 mL. The Idtotal concentration in each gellyfish is approximately 1.5×10⁻⁴ eq/L (6 μL beads/gellyfish).

Gellyfish blanks containing no beads (GFblank) are prepared following the same recipe as above (minus the beads) and deployed alongside GFbeads in all experiments to quantify Cu within the gel matrix that was not bound by iminodiacetate groups (CuGFblank).

Analysis of plasma and urine samples may also be carried out by contacting samples with gellyfish in buffer at a controlled temperature, washing, and then incubating in 10% nitric acid for a period of time, and then measuring copper by icp mass spectrometry.

### Example 4

An assay is provided that is capable of measuring chelatable copper in a subject. Copper may be measured, for example, from plasma or urine. The assay comprises: 1) immobilizing a copper antagonist to a solid matrix, 2) incubating, for example, plasma or urine with immobilized copper antagonist, 3) an optional stringency step, 4) rinsing non-specifically bound molecules from the matrix, 5) eluting copper, and 6) measuring copper levels.

Triethylenetetramine disuccinate, for example, is immobilized on Sepharose beads using cyanogen-bromide activated Sepharose beads, according to the manufacture's instructions. (CNBr-activated Sepharose 4B Amersham Biosciences). Briefly, 1-10 μmole/mL of triethylenetetramine disuccinate is added to the coupling buffer (0.1 M NaHCO₃, pH 8.3/0.5 M NaCl). The activated beads are added to the coupling buffer and then incubated, while rotating for one hour at room temperature. The beads are then washed once with coupling buffer. The beads are subsequently incubated for two hours in blocking buffer (0.1 M Tris-HCL pH 8.0) to block any remaining active groups. The beads are then washed with three cycles of alternating wash buffer (0.1 M acetate buffer, pH 4.0 and 0.1 M Tris-HCL, pH 8).

Once the immobilizing process is completed, the sepharose beads are packed into a column and the plasma or urine is passed through the column. Optionally, free ligands specific to non-copper metals that compete with copper for Triethylenetetramine disuccinate binding (e.g. ferrous or ferric cations), may be added to the plasma or urine prior to running on through the column. The column is rinsed with a suitable buffer, such as PBS pH 7.4 to remove non-specifically bound molecules. The column is then rinsed with a suitable low ionic strength buffer or deionized water. Copper is dissociated from the Sepharose beads using a suitable low pH buffer in which the pH is adjusted using hydrochloric acid. The eluate is collected and the pH readjusted to about 7.4 using a suitable bicarbonate or sodium phosphate buffer. Copper is measured using fluorescence spectrophotometry. A fluorescent dye, for example, such as Tetrakis-(4-sulfophenyl)-porphine (TSPP), is added to 1 mL elute. Each sample is read using a spectrometer. The concentration of copper can be determined by comparing each sample to a standard curve, for example.

Copper antagonists other than triethylenetetramine disuccinate may be used. For example, a copper antagonist precomplexed with a non-copper metal may be used, such as a triethylenetetramine precomplexed with calcium or another non-copper metal. Pentacoordinate copper antagonists may also be used. For example, a triethylenetetramine precomplexed with calcium or another non-copper metal and another complexing agent, such as, for example, chloride. Other copper antagonists may also be used, for example, d-penicillamine, diminoacetic acid and thiomolybdates.

Other solid matrices may also be used, including cellulose, and cellulose derivatives, polysaccharide hydrophilic polymers, including but not limited to cross-linked dextran derivates such as Sephadex, which can be purchased commercially from Amersham Biosciences; agarose and beaded agarose derivatives (available commercially from Amersham Biosciences; Bio-Rad Laboratories), polyacrylamide gels, beads or spheres (available from Bio-Gel, Bio-Rad Laboratories), and membranes, for example vivaspin metal chelate membranes and columns and cellulose membranes.

Other coupling methods are also known in the art and may include, for example, N-hydroxysuccinimide (NHS)-activated Sepharose 4 Fast Flow beads, activated CH Sepharose 4B, EAH Sepharose 4B (amine groups, epoxy-activated Sepharose 6B, EAH Sepharose 4B, activated Thiol Sepharose 4B, and Thiopropyl Sepharose 6B (all commercially available through Amersham Biosciences). Other fluorecent dyes include, but are not limited to Phen Green FL A (see Judd, et al., Proc SPIE-Int. Soc. Opt. Eng. 2388, 238 (1995)), Calcein (see Dean et al., Bioorganic Med. Chem. Let. 13, 1653-1656 (2003)), Fura-2, Fluo-4, and FuraZin-1.

### Example 5

This Example shows the utility of a copper antagonist to increase mRNA expression levels of EC-SOD.

All studies were approved by relevant ethics and regulatory committees. Rats were housed in individual cages under conditions of constant temperature (22° C.) and humidity. They were exposed to a 12:12-hour light-dark cycle and allowed unrestricted access to water and to standard rat chow.

Male Wistar rats, weighing about 220 g to about 250 g, were rendered diabetic by a single intravenous injection of streptozotocin (STZ; Sigma; 55 mg/kg bodyweight) in isotonic saline, pH 4.5, as described previously. Cooper et al., Diabetes 53:2501-2508 (2004). Age-matched control rats were injected with equal volumes of saline. Successful induction of diabetes was confirmed 24 hours after injection by elevated [glucose]_{blood} (>250 mg/dL; Glucometer Elite XL, Bayer, Elkhart, Ind.). Bodyweights and [glucose]_{blood} were monitored weekly for 16 weeks. Eight weeks after STZ injection, rats were assigned to three groups: untreated non-diabetic control; untreated diabetic; and triethylenetetramine-treated diabetic (administered as triethylenetetramine dihydrochloride, Fluka). Triethylenetetramine, at a dose of 20 mg/day/rat dissolved in the drinking water, was administered to diabetic rats for a further 8 weeks.

Sixteen weeks after STZ injection, rats were anesthetized, sacrificed and organs surgically removed thereafter. Aortas and cardiac left ventricles (LV) were either perfused for measurement of cardiac function or washed free of blood in DEPC-treated phosphate-buffered saline (PBS). Tissues were stored in RNAlater (Ambion) overnight at 4° C., and then at −80° C. for subsequent RNA isolation.

Measurement of cardiac function in rats. Cardiac function was determined as previously detailed. Cooper et al., supra (2004). Briefly, cardiac function was measured in isolated perfused working hearts. Rats were anesthetized and heparinized, and hearts were removed and then immersed in ice cold Krebs-Henseleit bicarbonate buffer (KHB). Working-mode perfusion was then established. Intra-chamber LV pressures, aortic pressure, and aortic and coronary flows were recorded, and maximum (−dP_{LV}/dt)ₘₑₐₙ values were determined at increasing levels of preload pressures (Powerlab 16s, ADI). Δ(−dP_{LV}/dt)ₘₑₐₙ values were calculated at different preload pressures by subtracting the value at 5 cmH₂O for each heart from subsequent values. Decreased (−dP_{LV}/dt)ₘₑₐₙ reflects increased stiffness of the LV wall, which may be associated with increased content and altered three-dimensional organization of fibrous connective tissue structures, such as those formed by collagen.

RNA isolation and cDNA synthesis. Approximately 100 mg of each tissue was sliced, and homogenized in 3 mL lysis buffer. Total RNA was isolated from aorta or LV using an RNA Midi Kit (Qiagen, Valencia, Calif.). RNA concentrations were determined spectrophotometrically using a Nanodrop apparatus, and RNA-integrity verified by agarose gel electrophoresis. One μg of total RNA was treated with RQ1 RNase-free DNase (Promega) at 37° C. for 30 minutes, and then reverse-transcribed with random hexamers and SuperScript™ III Reverse Transcriptase (Invitrogen).

Real-time quantitative PCR analysis. Gene expression was determined using real-time quantitative PCR (qPCR). A list of primers for each gene analyzed is below. Messenger RNA levels were compared by qPCR with an ABI Prism 7900 HT Sequence Detection System (Applied Biosystems, Foster City, Calif., USA). Reactions were prepared in the presence of the fluorescent dye SYBR green I for specific detection of double-stranded DNA. The levels of gene expression of the target sequence were normalized to those of an active endogenous control, 18S ribosomal RNA (r18S, Ambion). Varying sizes of oligonucleotides produce dissociation peaks at different melting temperatures. After PCR amplification, dissociation curves were constructed and PCR products were subjected to agarose gel electrophoresis to confirm formation of the specific PCR products. The threshold cycle (Ct) at which the fluorescent signal reaches a particular threshold value was used as a measure of gene expression. Ct values are approximate indicators of the abundance of a particular mRNA within tissues. The linear range of dilution for target genes and r18S showed a different slope, indicating different amplification efficiency for control and target genes, and a standard curve method was therefore used. Relative measurement of mRNA expression was performed as described in User Bulletin #2 (Applied Biosystems) using standard curves prepared from serially-diluted control cDNA samples.

EC-SOD primers:

Forward primer (5′-3′) GGCCCAGCTCCAGACTTGA
Reverse primer (5′-3′) CTCAGGTCCCCGAACTCATG

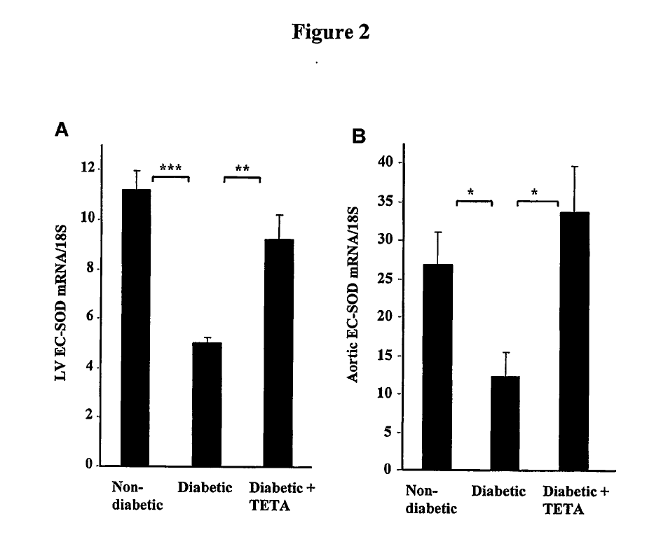

EC-SOD expression (LV and aorta). As shown in FIG. 2, mRNA expression levels of EC-SOD in LV and aorta from diabetic rats at 16 weeks were significantly decreased by 2.2- and 2.1-fold, respectively, when compared with those in non-diabetic control rats (FIG. 2A-B). However, 8-week treatment with the copper antagonist triethylenetetramine dihydrochloride significantly restored EC-SOD mRNA levels in these diabetic tissues by 2.8- and 1.8-fold, respectively (FIG. 2A-B).

### Example 6

This Example shows the utility of a copper antagonist to increase heparin sulfate levels, and describes the measurement of heparan sulfate in the left ventricle and aorta from control mice and diabetic mice treated with triethylenetetramine.

All studies were approved by relevant ethics and regulatory committees. Male Wistar rats were housed in individual cages under conditions of constant temperature (22° C.) and humidity. Rats were exposed to a 12:12-hour light-dark cycle and allowed unrestricted access to water and standard rat chow.

Rats weighing about 220 g to about 250 g, were rendered diabetic by a single intravenous injection of streptozotocin (STZ; Sigma; 55 mg/kg bodyweight) in isotonic saline, pH 4.5, as previously described in Cooper et al., Diabetes 53:2501-2508 (2004). Age-matched control rats were injected with equal volumes of saline. Successful induction of diabetes was confirmed 24 hours after injection by elevated blood glucose levels (>250 mg/dL; Glucometer Elite XL, Bayer, Elkhart, Ind.). Bodyweight and blood glucose were monitored weekly for 16 weeks.

Eight weeks after STZ injection, rats were assigned to four groups: untreated non-diabetic control; triethylenetetramine-treated non-diabetic; untreated diabetic; and triethylenetetramine-treated diabetic. Triethylenetetramine was administered as triethylenetetramine dihydrochloride, for eight weeks at a dose of 20 mg/day/rat. Sixteen weeks after STZ injection, the rats were anesthetized, sacrificed and the organs were surgically removed. Aortas and cardiac left ventricles (LV) were washed free of blood in DEPC-treated phosphate-buffered saline (PBS). Tissues were stored in RNAlater (Ambion) overnight at 4° C., and then at −80° C.

Heparan sulfate levels were measured using enzyme-linked immunosorbent assay (ELISA). Frozen aortic or LV tissue was homogenized in ice-cold PBS buffer. Homogenates were centrifuged at 13,000 g for 20 minutes at 4° C. Supernatants were isolated, and protein concentrations determined by BCA (Pierce). Heparan sulfate levels were measured as previously described in Yokoyama et al. Kidney International 56:650-658 (1999). Briefly, 10 μL of 20 mg/mL actinase E (Kaken Pharmaceuticals, Japan) was added to 100 μL supernatant and incubated at 55° C. (using a water bath) for 16-20 hours. After incubation, samples were boiled for 5 minutes, centrifuged at 3,000 g for 10 minutes, and then cooled. Supernatants were assayed for heparan sulfate by ELISA (Seikagalcu, Tokyo, Japan).

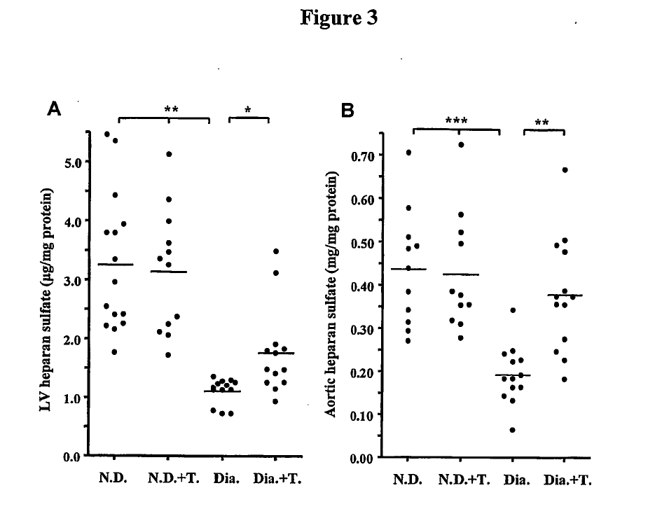

Heparan sulfate levels in both LV (FIG. 3A) and aorta (FIG. 3B) of diabetic rats were significantly lower than that of non-diabetic controls. Triethylenetetramine treatment significantly increased heparan sulfate levels in both tissues (FIG. 3A-B).

From the foregoing, it will be appreciated that, although specific embodiments of the invention have been described herein for the purpose of illustration, various modifications may be made without deviating from the spirit and scope of the invention. The foregoing description is intended to illustrate and not limit the scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the inventions pertain, and each such referenced document and material is hereby incorporated by reference to the same extent as if it had been incorporated by reference in its entirety individually or set forth herein in its entirety. The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent. Applicants reserve the right to physically incorporate into this specification any and all materials and information from any such patents, applications, publications, scientific articles, web sites, electronically available information, and other referenced materials or documents.

The specific methods and compositions described herein are representative of preferred embodiments and are exemplary and not intended as limitations on the scope of the inventions. Other objects, aspects, and embodiments will occur to those skilled in the art upon consideration of this specification, and are encompassed within the spirit of the inventions as defined by the scope of the claims. It will be readily apparent to one skilled in the art that varying substitutions and modifications may be made to the inventions disclosed herein without departing from the scope and spirit of the inventions. The inventions illustratively described herein suitably may be practiced in the absence of any element or elements, or limitation or limitations, which is not specifically disclosed herein as essential. Thus, for example, in each instance herein, in embodiments or examples of the present inventions, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically or otherwise expressly disclosed herein. Under no circumstances may the patent be interpreted to be limited by any statement made by any Examiner or any other official or employee of the Patent and Trademark Office unless such statement is expressly and specifically, without qualification or reservation, adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. Thus each is to be read as including the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group, which also form a part of the written description. It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention.

1. A method of determining response of a subject to a copper antagonist for treatment of a disease, disorder or condition, the method comprising: correlating (i) a measurement of copper in a sample from the subject with (ii) a hemoglobin A_{1c} measurement for the subject and/or a measurement of extracellular superoxide dismutase activity in a sample from the subject, and identifying therefrom the probability of response to said copper antagonist.

2. The method of claim 1, wherein both hemoglobin A_{1c} and a measurement of extracellular superoxide dismutase are correlated.

3. The method of claim 1, wherein a positive response probability is identified if (i) serum copper is at least about 14 μM and (ii) hemoglobin A_{1c} is at least about 8% and/or serum extracellular superoxide dismutase activity is at least about 1.5 times the upper limit of normal extracellular superoxide dismutase activity.

4. The method of claim 1, wherein a positive response probability is identified if (i) serum copper is at least about 14 μM, (ii) hemoglobin A_{1c} is at least about 8%, and (iii) serum extracellular superoxide dismutase activity is at least about 1.5 times the upper limit of normal extracellular superoxide dismutase activity.

5. The method of claim 1, wherein a positive response probability is identified if (i) serum copper is at least about 20 μM and (ii) hemoglobin A_{1c} is at least about 6 to about 8% and/or (iii) serum extracellular superoxide dismutase activity is at least about 1.5 times the upper limit of normal extracellular superoxide dismutase activity.

6. The method of claim 1, wherein extracellular superoxide dismutase activity is determined by measuring the amount of extracellular superoxide dismutase.

7. The method of claim 1, wherein said disease, disorder or condition is characterized in whole or in part by (a) hypercupremia and/or copper-related tissue damage and (b) one or more of hypertension, hyperlipidemia, impaired glucose tolerance, impaired fasting glucose, hyperglycemia, and insulin resistance, or predisposition to, or risk for, (a) and (b).

8. The method of claim 1, wherein said subject is a human.

9. The method of claim 1, wherein said disease, disorder or condition is selected from the group consisting of heart disease, glucose metabolism disorders, weight disorders, lipid disorders, and neurological disorders.

10. The method of claim 9, wherein said glucose metabolism disorder is selected from the group consisting of impaired glucose tolerance, impaired fasting glucose, prediabetes, type 1 diabetes, type 2 diabetes, insulin resistance, hyperglycemia, hyperinsulinemia, hyperamylinemia, and metabolic syndrome.

11. The method of claim 9, wherein said heart disease is selected from the group consisting of hypertension, atherosclerosis, heart failure, and cardiomyopathy.

12. The method of claim 9, wherein said weight disorder is obesity.

13. The method of claim 9, wherein said lipid disorder is selected from the group consisting of hyperlipidemia, hypertriglyceridemia, and hypercholesterolemia.

14. The method of claim 9, wherein said neurological disorder is selected from the group consisting of Alzheimer's disease, Huntington's Disease and Parkinson's disease.

15. A method for detecting the presence or risk of developing diabetic complications in a subject, said method comprising: correlating (i) a measurement of copper in a sample from the subject with (ii) a hemoglobin A_{1c} measurement for the subject and/or a measurement of extracellular superoxide dismutase activity in a sample from the subject, wherein elevated copper and elevated hemoglobin A_{1c} and/or extracellular superoxide dismutase activity correlates with the presence of or risk of developing diabetic complications.

16. The method of claim 15, wherein both hemoglobin A_{1c} and a measurement of extracellular superoxide dismutase are correlated with a copper measurement.

17. A method for detecting the presence or risk of developing heart disease in a human, said method comprising: correlating (i) a measurement of copper in a sample from the subject with (ii) a hemoglobin A_{1c} measurement for the subject and/or a measurement of extracellular superoxide dismutase activity in a sample from the subject, wherein elevated copper and elevated hemoglobin A_{1c} and/or extracellular superoxide dismutase activity correlates with the presence of or risk of developing heart disease.

18. The method of claim 17, wherein both hemoglobin A_{1c} and a measurement of extracellular superoxide dismutase activity are correlated with a copper measurement.

19. A method for detecting the presence or risk of developing Alzheimer's disease, Huntington's Disease or Parkinson's disease in a subject, said method comprising: correlating (i) a measurement of copper in a sample from the subject with (ii) a hemoglobin A_{1c} measurement for the subject and/or a measurement of extracellular superoxide dismutase activity in a sample from the subject, wherein elevated copper and elevated hemoglobin A_{1c} and/or extracellular superoxide dismutase activity correlates with the presence of or risk of developing diabetic complications.

20. The method of claim 19, wherein both hemoglobin A_{1c} and a measurement of extracellular superoxide dismutase activity are correlated with a copper measurement.

21. A method for evaluating a compound for use in the treatment of a disease involving copper, said method comprising: a) administering said compound to a test subject for a predetermined period of time; b) obtaining one or more copper measurements from said test subject; c) obtaining one or more measurements of extracellular superoxide dismutase activity from said test subject; and d) correlating a change in copper and extracellular superoxide dismutase with effectiveness of the compound.

22. The method of claim 21, further comprising obtaining one or more hemoglobin A_{1c} measurements.

23. The method of claim 21, wherein extracellular superoxide dismutase activity is determined by measuring the amount of extracellular superoxide dismutase.

24. The method of claim 21, wherein said disease, disorder or condition is selected from the group consisting of heart disease, glucose metabolism disorders, weight disorders, lipid disorders, and neurological disorders.

25. The method of claim 24, wherein said glucose metabolism disorder is selected from the group consisting of impaired glucose tolerance, impaired fasting glucose, prediabetes, type 1 diabetes, type 2 diabetes, insulin resistance, hyperglycemia, hyperinsulinemia, hyperamylinemia, and metabolic syndrome.

26. The method of claim 24, wherein said heart disease is selected from the group consisting of hypertension, atherosclerosis, heart failure, and cardiomyopathy.

27. The method of claim 24, wherein said weight disorder is obesity.

28. The method of claim 24, wherein said lipid disorder is selected from the group consisting of hyperlipidemia, hypertriglyceridemia, and hypercholesterolemia.

29. The method of claim 24, wherein said neurological disorder is selected from the group consisting of Alzheimer's disease, Huntington's Disease and Parkinson's disease.

30. A method of evaluating a subject for copper regulation therapy, which comprises:
obtaining at least one serum sample and/or at least one urine sample from said subject;
obtaining a hemoglobin A_{1c} measurement from said subject;
measuring copper concentration in a serum and/or urine sample from said subject; and,
identifying said subject as a candidate for copper regulation therapy where said subject has (i) a hemoglobin A_{1c} of at least about 8% and (ii) a serum copper concentration of at least about 14 μM and/or a urine copper concentration of at least about 100 nM.

31. The method of claim 30, wherein said subject has (i) serum copper of at least about 20 μM and (ii) hemoglobin A_{1c} of at least about 6 to about 8%.

32. The method of claim 30 further comprising measuring extracellular superoxide dismutase activity in a serum sample from said subject, and identifying said subject as a candidate for copper regulation therapy where said subject has elevated extracellular superoxide dismutase activity.

33. The method of claim 32, wherein extracellular superoxide dismutase activity is determined by measuring the amount of extracellular superoxide dismutase.

34. The method of claim 32 wherein said extracellular superoxide dismutase activity is at least about 1.5 times the upper limit of normal serum extracellular superoxide dismutase activity.

35. A method for identifying a subject as a candidate for copper regulation therapy, which comprises:
determining in said subject levels of (i) copper and (ii) one or more of elevated hemoglobin A_{1c} and extracellular superoxide dismutase activity; and,
identifying said subject as a candidate for copper regulation therapy based on elevated levels of (i) copper and (ii) hemoglobin A_{1c} and/or extracellular superoxide dismutase activity.

36. The method of claim 35, wherein extracellular superoxide dismutase activity is determined by measuring the amount of extracellular superoxide dismutase.

37. The method of claim 35, wherein the subject has elevated copper, elevated hemoglobin A_{1c}, and elevated extracellular superoxide dismutase activity.

38. The method of claim 35, wherein elevated copper is determined by obtaining a serum sample from said subject and measuring serum copper.

39. The method of claim 35, wherein elevated copper is determined by obtaining a urine sample from said subject and measuring urine copper.

40. The method of claim 35, wherein said subject is human.

41. A method for determining whether to initiate, continue, modify or terminate copper regulation therapy in a subject, which comprises measuring (a) copper and (b) one or more of hemoglobin A_{1c} and extracellular superoxide dismutase activity in said subject; and,
determining whether to initiate, continue, modify or terminate copper regulation therapy for treatment of a disease, disorder or condition in said subject based on the measurement of (i) copper and (ii) one or more of hemoglobin A_{1c} and extracellular superoxide dismutase activity.

42. A method of claim 41 wherein determination of whether to initiate, continue, modify or terminate copper regulation therapy for treatment of a disease, disorder or condition in said subject based on the measurement of (i) copper, (ii) hemoglobin A_{1c} and (iii) extracellular superoxide dismutase activity.

43. The method of claim 41, further comprising the step of initiating or continuing copper regulation therapy in said subject when said subject is determined to have (a) elevated copper and (b) one or more of elevated hemoglobin A_{1c} and elevated extracellular superoxide dismutase activity.

44. A method of claim 41 further comprising modifying a copper regulation therapy regimen for said subject based on the measurement of (i) copper and (ii) one or more of hemoglobin A_{1c} and extracellular superoxide dismutase activity.

45. The method of claim 41, further comprising modifying a copper regulation therapy regimen for said subject when said subject is determined to have (a) elevated copper and (b) one or more of elevated hemoglobin A_{1c} and elevated extracellular superoxide dismutase activity when compared to (i) at least one previous measurement of copper in said subject and (ii) at least one previous measurement of hemoglobin A_{1c} or extracellular superoxide dismutase activity or both in said subject.

46. The method of claim 41, further comprising modifying a copper regulation therapy regimen for said subject when said subject is determined to have (a) reduced levels of copper and (b) one or more reduced levels of hemoglobin A_{1c} and reduced extracellular superoxide dismutase activity when compared to (i) at least one previous measurement of copper in said subject and (ii) at least one previous measurement of hemoglobin A_{1c} or extracellular superoxide dismutase activity or both in said subject.

47. The method of claim 41, further comprising modifying a copper regulation therapy regimen to low dose copper therapy for said subject when said subject is determined to have (i) a serum copper concentration of less than about 14 μM and/or a urine copper concentration of less than about 100 nM; (ii) a hemoglobin A_{1c} of less than about 6 to less than about 8% and/or (iii) a extracellular superoxide dismutase activity of less than about 1.5 times the upper limit of normal.

48. The method of claim 41, further comprising the step of terminating copper regulation therapy in said subject when said subject is determined to have (i) a serum copper concentration of less than about 14 μM and/or a urine copper concentration of less than about 100 nM; (ii) a hemoglobin A_{1c} of less than about 6 to less than about 8% and/or (iii) a extracellular superoxide dismutase activity of less than about 1.5 times the upper limit of normal.

49. The method of claim 41, wherein said copper is measured from serum.

50. The method of claim 48, wherein said serum copper is at least about 14 μM.

51. The method of claim 41, wherein said copper is measured from urine.

52. The method of claim 51, wherein said urine copper is at least about 100 nM per liter.

53. The method of claim 51, wherein said urine copper is at least about 300 nM per liter.

54. The method of claim 51, wherein said urine copper is at least about 500 nM per liter.

55. The method of claim 41, wherein said extracellular superoxide dismutase activity is determined by measuring extracellular superoxide dismutase.

56. The method of claim 55, wherein said extracellular superoxide dismutase is at least about 1.5 times the upper limit of normal.

57. The method of claim 55, wherein said extracellular superoxide dismutase is at least about 40 Units per liter.

58. The method of any of claim 41, wherein said copper regulation therapy comprises administering a copper antagonist.

59. The method of any of claim 41, wherein said copper regulation therapy comprises administering a copper II antagonist.

60. The method of claim 59 wherein said copper II antagonist is a copper chelator.

61. The method of any of claim 41, wherein said copper regulation therapy comprises administering a trientine.

62. The method of claim 61 wherein said trientine is triethylenetetramine dihydrochloride or triethylenetetramine disuccinate.

63. The method of claim 59 wherein said copper II antagonist is pre-complexed with a non-copper metal ion.

64. The method of any of claim 41, wherein said copper regulation therapy comprises administering a thiomolybdate.

65. The method of any of claim 41 wherein said disease, disorder or condition is selected from the group consisting of heart disease, glucose metabolism disorders, weight disorders, lipid disorders, and neurological disorders.

66. The method of claim 65, wherein said glucose metabolism disorder is selected from the group consisting of impaired glucose tolerance, impaired fasting glucose, prediabetes, type 1 diabetes, type 2 diabetes, insulin resistance, hyperglycemia, hyperinsulinemia, hyperamylinemia, and metabolic syndrome.

67. The method of claim 65, wherein said heart disease is selected from the group consisting of hypertension, atherosclerosis, heart failure, and cardiomyopathy.

68. The method of claim 65, wherein said weight disorder is obesity.

69. The method of claim 65, wherein said lipid disorder is selected from the group consisting of hyperlipidemia, hypertriglyceridemia, and hypercholesterolemia.

70. The method of claim 65, wherein said neurological disorder is selected from the group consisting of Alzheimer's disease, Huntington's Disease and Parkinson's disease.

71. A method for qualifying a subject for copper regulation therapy, which comprises:
obtaining a hemoglobin A_{1c} measurement for said subject;
obtaining a serum copper concentration measurement for said subject; and,
identifying said subject as suitable for copper regulation therapy if said hemoglobin A_{1c} is at least about 8% and said serum copper is at least about 14 μM.

72. The method of claim 71, wherein said serum copper is at least about 16 μM.

73. The method of claim 71, wherein said serum copper is at least about 18 μM.

74. The method of claim 71, wherein said serum copper is at least about 20 μM.

75. The method of claim 71, wherein said hemoglobin A_{1c} is at least about 6 to about 8%.

76. The method of claim 71, further comprising measuring serum extracellular superoxide dismutase activity, and identifying said subject as suitable for copper regulation therapy if (i) hemoglobin A_{1c} is at least about 8%, (ii) serum copper is at least about 14 μM, and (iii) said serum extracellular superoxide dismutase activity is at least about 1.5 times the upper limit of normal extracellular superoxide dismutase activity. The method of claim 71, further comprising measuring serum extracellular superoxide dismutase activity, and identifying said subject as suitable for copper regulation therapy if (i) hemoglobin A_{1c} is at least about 8%, (ii) serum copper is at least about 14 μM, and (iii) said serum extracellular superoxide dismutase activity is at least about 40 Units per liter.

77. A method for qualifying a subject for copper regulation therapy, which comprises:
obtaining a hemoglobin A_{1c} measurement for said subject;
obtaining a urine copper measurement for said subject; and,
identifying said subject as suitable for copper regulation therapy if said hemoglobin A_{1c} is at least about 8% and said urine copper is at least about 100 nM.

78. The method of claim 77, wherein said urine copper is at least about 1.4 times the upper limit of normal urine copper.

79. The method of claim 77, wherein said urine copper is at least about 200 nM.

80. The method of claim 77, wherein said urine copper is at least about 300 nM.

81. The method of claim 77, wherein said urine copper is at least about 500 nM.

82. The method of claim 77, further comprising measuring serum extracellular superoxide dismutase activity, and identifying said subject as suitable for copper regulation therapy if (i) hemoglobin A_{1c} is at least about 8%, (ii) urine copper is at least about 100 nM, and (iii) said serum extracellular superoxide dismutase activity is at least about 1.5 times the upper limit of normal extracellular superoxide dismutase activity.

83. The method of claim 77, further comprising measuring serum extracellular superoxide dismutase activity, and identifying said subject as suitable for copper regulation therapy if (i) hemoglobin A_{1c} is at least about 8%, (ii) urine copper is at least about 100 nM, and (iii) said serum extracellular superoxide dismutase activity is at least about 40 Units per liter.

84. The method of claim 71 or 77, further comprising obtaining one or more total cholesterol, LDL-cholesterol, VLDL-cholesterol, oxidized LDL-cholesterol, HDL-cholesterol, and/or triglyceride measurement(s) for said subject.

85. The method of claim 84, further comprising identifying said subject as suitable for copper regulation therapy if said total cholesterol is at least about 200 mg/dL.

86. The method of claim 84, further comprising identifying said subject as suitable for copper regulation therapy if said LDL-cholesterol is at least about 130 mg/dL.

87. The method of claim 84, further comprising identifying said subject as suitable for copper regulation therapy if said VLDL-cholesterol is at least about 30 mg/dL.

88. The method of claim 84, further comprising identifying said subject as suitable for copper regulation therapy if said oxidized LDL-cholesterol is at least about 1.3 mg/dL.

89. The method of claim 84, further comprising identifying said subject as suitable for copper regulation therapy if said HDL-cholesterol is less than about 35 mg/dL.

90. The method of claim 84, further comprising identifying said subject as suitable for copper regulation therapy if said triglyceride is at least about 150 mg/dL.

91. The method of claim 84, further comprising identifying said subject as suitable for copper regulation therapy if the ratio of total cholesterol to HDL-cholesterol is greater than 6.4 and the subject is a man, or greater than 5.6 and the subject is a woman.

92. The method of claim 71 or 77, further comprising obtaining one or more homocysteine and/or highly sensitive C-reactive protein measurement(s) for said subject.

93. The method of claim 92, further comprising identifying said subject as suitable for copper regulation therapy if said homocysteine is at least about 11.4 μM/L.

94. The method of claim 92, further comprising identifying said subject as suitable for copper regulation therapy if said highly sensitive C-reactive protein is at least about 1.0 mg/L.

95. A method for assessing the therapeutic effect of copper regulation therapy in a subject, comprising:
obtaining a serum sample from said subject;
measuring hemoglobin A_{1c} and/or extracellular superoxide dismutase activity in a serum sample from said subject sample;
measuring serum copper concentration; and,
comparing said hemoglobin A_{1c} and/or extracellular superoxide dismutase activity and copper measurements with one or more previous hemoglobin A_{1c} and/or extracellular superoxide dismutase activity and copper measurements from said subject and assessing said therapeutic effect.

96. The method of claim 95, wherein extracellular superoxide dismutase activity but not hemoglobin A_{1c} is measured.

97. The method of claim 95, wherein both extracellular superoxide dismutase activity and hemoglobin A_{1c} are measured.

98. The method of claim 95, further comprising identifying said subject as suitable for copper regulation therapy if said homocysteine is at least about 11.4 μM/L.

99. A method for assessing the therapeutic effect of copper regulation therapy in a subject, comprising:
obtaining a serum sample from said subject;
measuring extracellular superoxide dismutase activity in said serum sample; and,
determining the effect of said copper regulation therapy on extracellular superoxide dismutase activity in said subject.

100. The method of claim 99, further comprising measuring total serum copper or total urine copper or both in said subject.

101. The method of claim 99, further comprising measuring hemoglobin A_{1c} in said subject.

102. The method of claim 99 further comprising identifying said subject as suitable for copper regulation therapy if said homocysteine is at least about 11.4 μM/L.

103. The method of claim 99 wherein intravascular consumption of NO is suppressed.

104. The method of claim 99, wherein vascular superoxide production is lowered.

105. The method of claim 99, wherein physiological vasodilatation is enhanced.

106. An assay for measuring chelatable copper in a sample comprising immobilizing a copper antagonist to a solid matrix; incubating said sample with said immobilized copper antagonist; rinsing non-specifically bound molecules from the solid matrix; eluting copper; and measuring copper levels using fluorescent spectrophotometery.

107. The assay of claim 106, further comprising an additional stringency step wherein a sample is incubated with a free ligand specific for non-copper metals.

108. The assay of claim 106 wherein the sample is a urine sample.

109. The assay of claim 106 wherein the sample is a plasma sample.

110. The assay of claim 106 wherein the sample is a serum sample.

111. A kit comprising an assay of claim 106 and instructions for its use.

112. A method of evaluating a subject for copper regulation therapy, which comprises obtaining at least one serum sample and/or at least one urine sample from said subject; obtaining a hemoglobin A_{1c} measurement from said subject; measuring homocysteine concentration in a serum and/or urine sample from said subject; and, identifying said subject as a candidate for copper regulation therapy where said subject has (i) elevated hemoglobin A_{1c} of at least about 8% and (ii) elevated serum or urine homocysteine.

113. The method of claim 112 further comprising measuring extracellular superoxide dismutase activity in a serum sample from said subject, and identifying said subject as a candidate for copper regulation therapy where said subject has elevated extracellular superoxide dismutase activity.

114. The method of claim 112, wherein extracellular superoxide dismutase activity is determined by measuring the amount of extracellular superoxide dismutase.


SEQUENCE LISTING (2 sequences)

SEQ ID NO: 1 - DNA, 19 bp, ARTIFICIAL
  Synthetic - made in laboratory
ggcccagctccagacttga

SEQ ID NO: 2 - DNA, 20 bp, ARTIFICIAL
  Synthetic - made in laboratory
ctcaggtccccgaactcatg